SEQUENCE LISTING (15 sequences)

SEQ ID NO: 1 - DNA, 2061 bp, Artificial Sequence
  Description of Artificial Sequence Synthetic polynucleotide
attctgaaatgagctgttgacaattaatcatccggctcgtataatgtgtggaaattgtgagcggataacaattaggaggttaattaacaatgagtcacatccagagagaaactagttgttcccgacctcgtttgaatagcaatatggatgcagatctgtacggatataaatgggcgcgagataacgtaggccaatctggggccactatttatcggttatatggcaaaccagatgctcccgaactgtttctcaaacatggcaaagggtctgtggccaatgatgttaccgatgaaatggtgcggttgaactggttgacagaatttatgcccctcccgaccatcaaacattttatcaggactccagacgatgcatggctattaactacggccattcctgggaaaactgcctttcaggtgttggaagaatatcccgattctggtgagaatatcgtcgatgcgttagcggtttttctaagacgtctacatagcattcccgtttgcaattgtccctttaattcggaccgggtgttccgcttggcgcaggctcagtcccggatgaataacggtttggtagatgcctcggactttgatgatgaacggaacggctggcccgttgaacaggtttggaaagagatgcataagctgctgcccttctcccccgacagcgttgttactcatggagatttttctctcgataatctgattttcgacgaaggcaagctaattggctgtatcgatgtgggacgggtagggattgcggaccggtatcaagacctagcaattttgtggaactgcctaggtgaattttcccccagcctacaaaaacggctgtttcaaaaatacggaatcgataatcccgacatgaacaaattacaatttcatctgatgctagatgagttctttcatatgacgcgcagcagtaaggccttggtccaactagctgatctatccgaacaagtaaaaaacgtggtggaatttgattttgacaagtatatgcactccaaggccattgcggttaatgaggccttagataaagttattcccccccgctatcctcaaaaaatctatgaaagtatgcgctattccctcctagccggcgggaagagggttcgaccaattttatgtattgcggcctgtgagctaatgggggggactgaggaacttgccatgcctacggcttgtgccatcgagatgattcacactatgagtttgattcatgacgatttgccctatattgataacgatgatttgcgtcgcggtaagcctaccaaccacaaagtttttggtgaagacacggcgatcattgctggcgatgcattattgtcattggcctttgaacatgtagccgtgagcaccagtcgtaccctaggtactgacattattttacggttgctatccgaaattggacgcgccacaggaagtgagggcgtgatgggtggtcaagtggtggatattgaaagcgaaggtgatcccagtatagacttagaaacgctggaatgggtccatattcataaaacggctgtgttgttggaatgcagtgtcgtgtgtggcgcaattatggggggtgccagcgaggacgacatcgagcgtgctagacggtacgctcgctgtgtaggattgcttttccaagttgtcgatgatattttggatgtaagccagtcctcggaagaactcggaaagactgctgggaaagatttgatttctgacaaagccacctatcccaaactcatgggtttggaaaaagcgaaggaatttgccgatgaattactgaaccgtggaaaacaggaacttagttgttttgatcctaccaaagcagcacctctatttgcgttagcagactacattgcatctcgtcagaattaaggatcctccttggtgtaatgccaactgaataatctgcaaattgcactctccttcaatggggggtgctttttgcttgactgagtaatcttctgattgctgatcttgattgccatcgatcgccggggagtccggggcagttaccattagagagtctagagaattaatccatcttcgatagaggaattatgggggaagaacc

SEQ ID NO: 2 - protein, 590 aa, Artificial Sequence
  Description of Artificial Sequence Synthetic polypeptide
MSHIQRETSCSRPRLNSNMDADLYGYKWARDNVGQSGATIYRLYGKPDAPELFLKHGKGSVANDVTDEMVRLNWLTEFMPLPTIKHFIRTPDDAWLLTTAIPGKTAFQVLEEYPDSGENIVDALAVFLRRLHSIPVCNCPFNSDRVFRLAQAQSRMNNGLVDASDFDDERNGWPVEQVWKEMHKLLPFSPDSVVTHGDFSLDNLIFDEGKLIGCIDVGRVGIADRYQDLAILWNCLGEFSPSLQKRLFQKYGIDNPDMNKLQFHLMLDEFFHMTRSSKALVQLADLSEQVKNVVEFDFDKYMHSKAIAVNEALDKVIPPRYPQKIYESMRYSLLAGGKRVRPILCIAACELMGGTEELAMPTACAIEMIHTMSLIHDDLPYIDNDDLRRGKPTNHKVFGEDTAIIAGDALLSLAFEHVAVSTSRTLGTDIILRLLSEIGRATGSEGVMGGQVVDIESEGDPSIDLETLEWVHIHKTAVLLECSVVCGAIMGGASEDDIERARRYARCVGLLFQVVDDILDVSQSSEELGKTAGKDLISDKATYPKLMGLEKAKEFADELLNRGKQELSCFDPTKAAPLFALADYIASRQN

SEQ ID NO: 3 - DNA, 8442 bp, Artificial Sequence
  Description of Artificial Sequence Synthetic polynucleotide
ctcgagaagagtccctgaatatcaaaatggtgggataaaaagctcaaaaaggaaagtaggctgtggttccctaggcaacagtcttccctaccccactggaaactaaaaaaacgagaaaagttcgcaccgaacatcaattgcataattttagccctaaaacataagctgaacgaaactggttgtcttcccttcccaatccaggacaatctgagaatcccctgcaacattacttaacaaaaaagcaggaataaaattaacaagatgtaacagacataagtcccatcaccgttgtataaagttaactgtgggattgcaaaagcattcaagcctaggcgctgagctgtttgagcatcccggtggcccttgtcgctgcctccgtgtttctccctggatttatttaggtaatatctctcataaatccccgggtagttaacgaaagttaatggagatcagtaacaataactctagggtcattactttggactccctcagtttatccgggggaattgtgtttaagaaaatcccaactcataaagtcaagtaggagattaattcagagctgttgacaattaatcatccggctcgtataatgtgtggaaattgtgagcggataacggaattaggaggttaattaaatgggaaaaaactataaatccctggacagtgtcgtcgcgtctgattttattgcattgggcattaccagtgaagtagcagagaccctgcatgggcgactagctgaaatcgtttgtaattacggagcagcgactccacaaacgtggatcaacatcgcgaatcatatcttaagtccagatctgcctttctccttgcaccagatgttgttttacggatgttataaggattttgggcccgcgcctcctgcttggatcccagaccctgagaaggtaaaaagcaccaacttgggagcattactggagaagcgtggcaaagagttcttaggagtaaagtacaaagacccaatttctagctttagtcactttcaagaatttagtgttcggaatcctgaagtgtattggcgtacagtattaatggatgaaatgaagatcagtttttctaaggacccagaatgtatcctacgtcgagatgatatcaacaatccaggaggtagtgaatggctacctggaggttacttgaacagtgctaagaactgtttaaatgtcaactctaataaaaagttgaacgacactatgatcgtctggcgcgacgaaggcaacgatgatttaccattgaacaaactcacgttagatcagttacggaaacgtgtgtggttagttgggtacgcattagaagagatgggtttggagaaaggttgtgccattgctattgacatgccaatgcacgtcgacgcggtcgttatctatttggctatcgtactagccggatatgtagttgtgtctatcgcggactctttcagtgcccccgagatcagtactcgtctgcgactatccaaggcgaaggctatcttcacgcaggatcacatcattcggggcaaaaaacgaattcctttgtactctcgcgtggttgaggcgaaaagccctatggctatcgtgattccgtgcagcggaagcaatattggtgcagaactacgagatggagacatcagttgggactatttcttagaacgagctaaagagttcaaaaattgtgaattcacagcgcgagaacaaccagtggacgcttatacaaacatcttattttctagtggaacaacaggagaacctaaagcaatcccttggactcaagcgacccctctaaaagctgccgcggatggatggagccatctagacattcgtaagggtgatgtcattgtttggccgacgaatctgggttggatgatgggtccttggctagtttacgcatctctcctaaacggcgccagtatcgctctctacaacgggtctcctctggttagcggattcgcaaaattcgtgcaggacgctaaagtgactatgctaggagtggtcccttctatcgtgcgtagctggaagagcacaaactgcgtctctggatatgattggtctaccatccggtgctttagttcttccggagaagccagcaatgttgatgagtacctgtggttaatgggccgggcaaattacaaaccagttattgagatgtgtggaggaacagaaattgggggagcgttctctgcggggagtttcttgcaagcccaatccctctccagttttagcagtcaatgtatgggctgcactttatacattttggacaagaacggttacccaatgccgaaaaacaaaccgggcattggtgaattagcactaggtccagtaatgttcggagctagtaagacactgttaaatggcaaccatcacgatgtctatttcaaggggatgcccacattaaatggtgaggtcttacgtcgtcacggggacattttcgagttaacctctaatgggtattatcacgctcacgggcgagcggatgacacgatgaacatcggagggattaaaatcagttccatcgaaattgagcgtgtgtgcaatgaggtagacgatcgggtattcgagacaacggccatcggggtgccgcccctcggagggggacccgaacaattggtaattttttttgtcctgaaggattccaacgataccacaatcgacttgaatcagttgcgcctcagcttcaacttaggcttgcagaagaagctaaacccactcttcaaggttacgcgggttgtaccactgtctagcctccctcggactgctacgaataaaatcatgcgccgagtactccgccaacaattcagtcacttcgaataaggaattaggaggttaattaaatgaatcacttgcgagcggaaggtcccgctagtgtactcgctattgggactgccaacccagaaaatattttactccaggatgagttcccggattattacttccgagtcacaaagagcgaacacatgacgcagttaaaagagaagttccgcaaaatctgtgacaagtctatgattcgcaaacgcaattgctttttgaatgaagaacatctgaagcagaatccacgtctggttgagcacgagatgcagactttagacgctcgacaggacatgctagtcgtggaagtcccgaaactgggtaaagacgcgtgtgccaaggccattaaggaatggggtcaacctaagagtaagatcacccatctcatttttaccagtgcgtccacgacagacatgcctggagctgactaccattgtgccaagctcctaggactatctccatctgtgaaacgggtaatgatgtatcagctaggatgttatggtggggggactgtgttacgtatcgcaaaggatatcgcggagaataacaagggggctcgcgtcctagccgtttgctgcgacattatggcgtgcctctttcggggaccctccgagagcgacttggagctattagtaggccaagcgatctttggagatggggccgctgctgttattgttggcgctgaacccgatgagagtgtaggtgagcgcccaattttcgagttggtctccacgggtcagacaattctccccaacagtgaaggcacaattgggggacatatccgggaggcaggactgatctttgacctacataaggacgtcccgatgctcatttctaacaacattgaaaagtgcctgattgaagcgttcaccccaatcggcattagtgattggaatagtatcttctggattactcatcccggaggtaaagccattctagataaggtggaagaaaagttacacttaaagtccgacaagtttgtcgatagtcgtcacgtgctgagcgagcatgggaatatgagtagctctacggttttgttcgttatggacgaattacgaaagcgcagcttggaggagggaaaaagcacgacaggggatggatttgagtggggagttctctttggatttggtcccgggctgacagtagagcgcgtggtggtgcgctccgtgccgattaagtgaggaattaggaggttaattaaatggccgtaaagcacctgattgtattgaaattcaaagatgagatcacggaggcgcagaaggaggagtttttcaagacgtacgtgaacctagtgaatatcatcccggcgatgaaggatgtctattggggtaaagatgtaactcagaaaaacaaggaagaaggttacacccatattgttgaagtcacattcgaaagtgtagagacgatccaagattatattattcatccggctcacgttggatttggagacgtgtatcgttctttttgggagaagttgttaatcttcgactacaccccccgcaaatagggaattaggaggttaattaaatgggcttaagctctgtatgcactttcagtttccaaaccaattatcatacgctcctaaacccccacaataacaacccaaaaacatccttgttgtgctatcgacaccctaaaacgccaatcaagtattcttataacaattttccctccaaacactgctccactaagagcttccacctacaaaacaaatgtagcgaatccttgtccatcgccaagaactccattcgagcagcaaccaccaatcagacagagccacctgagagcgacaatcatagcgtcgcgactaagatcctaaatttcgggaaagcatgctggaaactacaacgaccatacacgatcatcgcgttcaccagttgcgcttgcggtttatttggtaaggaattgctccataataccaacctgatttcctggagtctgatgttcaaagcattttttttcttggtggccatcctatgtatcgcgtcttttacaacaaccatcaatcagatctatgacctccacatcgatcgcattaacaagccagacctcccattagcgtctggtgaaatctctgtcaacaccgcctggattatgagcattattgtagcactgtttgggctaatcattacaatcaagatgaagggtggacccctctacatttttggctattgcttcggaatctttggtggcattgtttacagcgtaccaccgtttcggtggaagcagaatcccagtaccgctttcctattgaactttctggcccacatcatcaccaactttacgttttactacgcaagtcgggcggcactgggcctcccattcgagctgcgacccagttttacgtttctcttagcgttcatgaaaagcatgggaagcgctctcgccctgattaaggatgcctccgacgtggaaggcgacacaaagttcggtatttctacattagcaagcaagtatggttcccgtaacctaacactcttttgttctggaattgtgttactaagttatgtagcagctattctggcaggtatcatttggccccaggccttcaatagcaatgttatgctgttatctcatgcgatcctcgccttctggttaatcctacagacacgggactttgccctcactaattacgatcccgaggcgggccgacgtttttacgagttcatgtggaagctatactatgcagagtacctcgtgtacgtgtttatttaaggaattaggaggttaattaaatgaagtgttctactttctctttctggtttgtctgcaagatcatttttttcttcttctctttcaatattcagacaagtattgcgaatccccgggagaactttttaaagtgttttagccaatatatccctaataatgctaccaatttaaaattagtatacacccaaaacaaccccctatacatgtccgttctcaatagcacaattcataacttgcgcttcacaagcgatacaacaccgaagcccctagttatcgtaaccccgagccacgtttctcacattcagggaaccattctctgcagtaaaaaggtgggtttgcagatccggactcggtctgggggtcatgacagtgagggtatgtcttacattagccaggtgccctttgtgatcgtcgacttacggaacatgcgctctattaagattgatgtccatagccaaaccgcgtgggtagaggccggagcaaccctgggtgaagtgtattactgggtaaatgagaaaaacgagaacttaagtctggcagctggatactgtccaaccgtctgcgcggggggtcatttcggagggggaggctacggcccactcatgcgtaattatgggttggcggctgacaacattattgatgctcacttagttaacgtgcacggtaaagtactggatcggaaatccatgggggaagatctattttgggccttacgaggaggaggagctgagtctttcggcattatcgtcgcgtggaaaattcggttagtcgcggtacccaagtctacgatgttttccgtgaaaaaaattatggagatccacgaactcgtgaagctagtcaacaagtggcagaatattgcttataagtacgacaaggatctgttattgatgacgcatttcatcacacgaaatatcacagacaatcaaggtaaaaacaagactgctatccacacctactttagctccgttttcttaggcggggtggattccctggtcgatctaatgaataaaagtttccccgaactaggcattaaaaagacagattgtcgtcaattatcttggattgacactattattttctatagcggcgtggtaaactatgacacggataactttaataaggagatcttgttggatcgcagtgcgggacagaacggcgcgtttaagattaagttggattatgtaaagaagcccattccagagtctgttttcgtacagatcttagaaaaattatatgaggaggatatcggggccggtatgtatgccttgtatccgtacggtggaatcatggacgaaatcagcgagagtgccattccgttcccccatcgagccggaattttgtatgaattatggtacatctgcagctgggagaaacaagaagataacgagaaacacttgaactggattcgtaacatctataatttcatgactccgtatgtcagtaaaaaccctcggttggcttacctaaattaccgtgacctcgatattggaattaacgaccctaagaatccaaacaattacactcaagcccggatttggggggagaaatattttggcaagaacttcgatcgattggtaaaggtcaagactctcgtagatcctaataacttttttcgtaacgaacaatctatcccccctctgcctcgtcatcggcattagggaattaggaggttaattaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtgatttttttaaggcagttattggtgcccttaaacgcctggggatccgctattttgttaattactatttgagctgagtgtaaaataccttacttactcaaaagcattaactaaccataacaatgactaatctctttttttgattgaactccaaactagaatagccatcgagtcagtccatttagttcattattagtgaaagtttgttggcggtgggttatccgttgataaaccaccgtttttgtttgggcaaagtaacgatttgatgcagtgatgggtttaaagataatcccgtttgaggaaatcctgcaggacgacgggaactttaacctgaccgctgctgggttcgtaataattttctaaaattgccgccatggtgcgcccgatcgccaaaccggaaccgttgagagtgtgaacaaattgggtgccttttttgcccttttccttgtagcgaatgttggcccgacgggcttggaaatcgtggaagttagaacaactggaaatttcccggtaggtgttagccgatggtaaccaaacttccaagtcgtagcatttagccgctccaaaacctaaatcaccggtacataattccaccactgagctc

SEQ ID NO: 4 - protein, 720 aa, Cannabis sativa
MGKNYKSLDSVVASDFIALGITSEVAETLHGRLAEIVCNYGAATPQTWINIANHILSPDLPFSLHQMLFYGCYKDFGPAPPAWIPDPEKVKSTNLGALLEKRGKEFLGVKYKDPISSFSHFQEFSVRNPEVYWRTVLMDEMKISFSKDPECILRRDDINNPGGSEWLPGGYLNSAKNCLNVNSNKKLNDTMIVWRDEGNDDLPLNKLTLDQLRKRVWLVGYALEEMGLEKGCAIAIDMPMHVDAVVIYLAIVLAGYVVVSIADSFSAPEISTRLRLSKAKAIFTQDHIIRGKKRIPLYSRVVEAKSPMAIVIPCSGSNIGAELRDGDISWDYFLERAKEFKNCEFTAREQPVDAYTNILFSSGTTGEPKAIPWTQATPLKAAADGWSHLDIRKGDVIVWPTNLGWMMGPWLVYASLLNGASIALYNGSPLVSGFAKFVQDAKVTMLGVVPSIVRSWKSTNCVSGYDWSTIRCFSSSGEASNVDEYLWLMGRANYKPVIEMCGGTEIGGAFSAGSFLQAQSLSSFSSQCMGCTLYILDKNGYPMPKNKPGIGELALGPVMFGASKTLLNGNHHDVYFKGMPTLNGEVLRRHGDIFELTSNGYYHAHGRADDTMNIGGIKISSIEIERVCNEVDDRVFETTAIGVPPLGGGPEQLVIFFVLKDSNDTTIDLNQLRLSFNLGLQKKLNPLFKVTRVVPLSSLPRTATNKIMRRVLRQQFSHFE

SEQ ID NO: 5 - protein, 385 aa, Cannabis sativa
MNHLRAEGPASVLAIGTANPENILLQDEFPDYYFRVTKSEHMTQLKEKFRKICDKSMIRKRNCFLNEEHLKQNPRLVEHEMQTLDARQDMLVVEVPKLGKDACAKAIKEWGQPKSKITHLIFTSASTTDMPGADYHCAKLLGLSPSVKRVMMYQLGCYGGGTVLRIAKDIAENNKGARVLAVCCDIMACLFRGPSESDLELLVGQAIFGDGAAAVIVGAEPDESVGERPIFELVSTGQTILPNSEGTIGGHIREAGLIFDLHKDVPMLISNNIEKCLIEAFTPIGISDWNSIFWITHPGGKAILDKVEEKLHLKSDKFVDSRHVLSEHGNMSSSTVLFVMDELRKRSLEEGKSTTGDGFEWGVLFGFGPGLTVERVVVRSVPIKY

SEQ ID NO: 6 - protein, 101 aa, Cannabis sativa
MAVKHLIVLKFKDEITEAQKEEFFKTYVNLVNIIPAMKDVYWGKDVTQKNKEEGYTHIVEVTFESVETIQDYIIHPAHVGFGDVYRSFWEKLLIFDYTPRK

SEQ ID NO: 7 - protein, 395 aa, Cannabis sativa
MGLSSVCTFSFQTNYHTLLNPHNNNPKTSLLCYRHPKTPIKYSYNNFPSKHCSTKSFHLQNKCSESLSIAKNSIRAATTNQTEPPESDNHSVATKILNFGKACWKLQRPYTIIAFTSCACGLFGKELLHNTNLISWSLMFKAFFFLVAILCIASFTTTINQIYDLHIDRINKPDLPLASGEISVNTAWIMSIIVALFGLIITIKMKGGPLYIFGYCFGIFGGIVYSVPPFRWKQNPSTAFLLNFLAHIITNFTFYYASRAALGLPFELRPSFTFLLAFMKSMGSALALIKDASDVEGDTKFGISTLASKYGSRNLTLFCSGIVLLSYVAAILAGIIWPQAFNSNVMLLSHAILAFWLILQTRDFALTNYDPEAGRRFYEFMWKLYYAEYLVYVFI

SEQ ID NO: 8 - protein, 544 aa, Cannabis sativa
MKCSTFSFWFVCKIIFFFFSFNIQTSIANPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 9 - DNA, 1635 bp, Cannabis sativa
atgaactgtagcgcatttagtttctggttcgtgtgtaagatcatttttttctttttatcttttcacattcagatttctatcgctaatccgcgcgaaaatttcctcaaatgctttagtaagcacatcccaaacaacgttgcgaatcccaaactggtctacacgcagcacgatcagctctacatgtctatcctgaatagcacaatccagaacttacggttcatctctgatacaacgccaaagcctttagtgattgttacaccgagcaacaattctcatatccaagccacaattttgtgcagtaaaaaggttgggttgcaaatccgaacgcgcagcgggggacacgacgcagagggtatgagttacatttctcaggtccccttcgttgttgtggatctacggaatatgcactccatcaagattgacgtacacagtcagaccgcttgggtcgaagccggagcaaccttaggcgaggtctactattggattaatgagaaaaacgagaacctctctttccctggtggatattgtcctactgtaggtgtcggagggcatttcagtggcggaggctatggggctctcatgcgcaattatggcttggccgcggacaatatcattgacgctcatctcgtgaacgtcgacggtaaggtactcgatcgtaaaagcatgggtgaggatctcttctgggctattcgaggtggtggaggagagaacttcggaattatcgcagcctggaaaattaagttagttgcggtccccagtaaaagcacaatctttagcgtcaaaaagaacatggaaattcatggactcgtaaagctctttaataaatggcagaacattgcatacaaatatgacaaagacctagtgttgatgacccattttattactaaaaatattacggataaccacgggaagaacaagacaacagtacatggttactttagcagcatcttccacggtggggtcgattctctagtagacctgatgaataagtcctttccggaactaggcatcaagaaaactgactgcaaagaattttcctggatcgacacgactatcttctatagtggagtagtaaactttaatacagcaaacttcaaaaaagaaatcctgctagatcgatccgcggggaagaagactgcatttagcattaagctggactatgtaaagaaacccattccggagacagccatggttaaaattttggagaaattgtacgaagaggacgtcggagccggcatgtacgtcctctatccttatggcgggattatggaggaaatcagtgagtccgctatccctttcccccaccgtgcgggtatcatgtacgagttatggtacaccgcgtcctgggaaaagcaggaggacaacgagaaacacatcaactgggtccgttccgtgtacaattttaccaccccttatgtttctcaaaatccgcgactcgcctatttaaactatcgtgacctggacctggggaaaacaaaccacgcgagtcccaataactacacgcaagcacgaatctggggtgaaaagtactttggtaagaatttcaatcgactggttaaagttaagacaaaagtcgatcctaacaatttcttccgaaatgagcaatctattccgcccttgcctcctcatcaccac

SEQ ID NO: 10 - protein, 545 aa, Cannabis sativa
MNCSAFSFWFVCKIIFFFLSFHIQISIANPRENFLKCFSKHIPNNVANPKLVYTQHDQLYMSILNSTIQNLRFISDTTPKPLVIVTPSNNSHIQATILCSKKVGLQIRTRSGGHDAEGMSYISQVPFVVVDLRNMHSIKIDVHSQTAWVEAGATLGEVYYWINEKNENLSFPGGYCPTVGVGGHFSGGGYGALMRNYGLAADNIIDAHLVNVDGKVLDRKSMGEDLFWAIRGGGGENFGIIAAWKIKLVAVPSKSTIFSVKKNMEIHGLVKLFNKWQNIAYKYDKDLVLMTHFITKNITDNHGKNKTTVHGYFSSIFHGGVDSLVDLMNKSFPELGIKKTDCKEFSWIDTTIFYSGVVNFNTANFKKEILLDRSAGKKTAFSIKLDYVKKPIPETAMVKILEKLYEEDVGAGMYVLYPYGGIMEEISESAIPFPHRAGIMYELWYTASWEKQEDNEKHINWVRSVYNFTTPYVSQNPRLAYLNYRDLDLGKTNHASPNNYTQARIWGEKYFGKNFNRLVKVKTKVDPNNFFRNEQSIPPLPPHHH

SEQ ID NO: 11 - DNA, 1590 bp, Cannabis sativa
atgaattgtagcacgttcagcttctggttcgtatgtaaaattatcttttttttcctcagttttaatatccaaatctctattgctaacccccaggagaatttcctcaagtgtttcagcgagtacattcctaacaaccctgctccaaaatttatctacacgcaacacgatcaattgtatatgagtgttttaaattccaccatccaaaacttgcgttttacctctgacactacaccaaagcctctcgtcattgtgacgccgagtaatgttagtcatattcaggcgagtattctctgctctaaagttggactccaaatccgcacgcgtagcggcggtcacgatgcggaagggttatcctacattagccaggtgcctttcgctattgttgacttgcgtaatatgcatacagtagtagacattcattcccagacggccgtggaggcaggcgcgacgttgggggaagtttactactggattaatgaaatgaatgaaaatttcagtttccctggaggttactgtccaactgttggagttggaggtcatttttccggaggaggatacggagcgttaatgcggaattacggattagcagcagataatatcatcgacgctcatctagtaaatgtagacggaaaagtattggaccgaaagagtatgggtgaggacttgttctgggctattcgagggggcgggggcgaaaacttcggtatcatcgcagcctgtatcaagctctgggtacccagtaaggccactattttctctgtcaaaaagaacatggagattcacggtctcgtgaagttatttaacaaatggcaaaatattgcctactacgataaagacttgatgttgacgacgcatttccgcacacgcaacattaccgacaaccatgggaataaaacaactgtacacggctatttttctagtatcttcctcgggggcgtagactccctcgtcgatttgatgaataaaagtttcccagaactgggtatcaaaactgactgtaaagaactgtcctggattgataccacgattttctattccggctggtataatacagcctttaagaaagaaattttactggatcgctctgcgggtaaaaagacggctttcagcatcaaactcgactacgttaaaaagctcattccggaaaccgctatggttaaaatcctagagttatacgaagaagaggttggcgtaggcatgtatgtactctacccatacggtggtattatggatgaaatctccgaatccgcaattccatttccccatcgcgcgggtatcatgtatgaactgtatacggcgactgagaaacaggaagacaacgaaaagcacatcaactgggtgcggtccgtctataactttaccaccccttatgtaagtcagaacccgcggctggcatatctaaattatcgggacctggatctaggcaaaacgaaccccgagtctccgaataactatactcaggcgcggatctggggggagaaatactttgggaaaaactttaaccgactcgtaaaggtaaaaaccaaggccgacccgaacaacttcttccgcaacgaacaatctattcccccactccccccacgccatcac

SEQ ID NO: 12 - protein, 530 aa, Cannabis sativa
MNCSTFSFWFVCKIIFFFLSFNIQISIANPQENFLKCFSEYIPNNPAPKFIYTQHDQLYMSVLNSTIQNLRFTSDTTPKPLVIVTPSNVSHIQASILCSKVGLQIRTRSGGHDAEGLSYISQVPFAIVDLRNMHTVVDIHSQTAVEAGATLGEVYYWINEMNENFSFPGGYCPTVGVGGHFSGGGYGALMRNYGLAADNIIDAHLVNVDGKVLDRKSMGEDLFWAIRGGGGENFGIIAACIKLWVPSKATIFSVKKNMEIHGLVKLFNKWQNIAYYDKDLMLTTHFRTRNITDNHGNKTTVHGYFSSIFLGGVDSLVDLMNKSFPELGIKTDCKELSWIDTTIFYSGWYNTAFKKEILLDRSAGKKTAFSIKLDYVKKLIPETAMVKILELYEEEVGVGMYVLYPYGGIMDEISESAIPFPHRAGIMYELYTATEKQEDNEKHINWVRSVYNFTTPYVSQNPRLAYLNYRDLDLGKTNPESPNNYTQARIWGEKYFGKNFNRLVKVKTKADPNNFFRNEQSIPPLPPRHH

SEQ ID NO: 13 - DNA, 8445 bp, Artificial Sequence
  Description of Artificial Sequence Synthetic polynucleotide
ctcgagaagagtccctgaatatcaaaatggtgggataaaaagctcaaaaaggaaagtaggctgtggttccctaggcaacagtcttccctaccccactggaaactaaaaaaacgagaaaagttcgcaccgaacatcaattgcataattttagccctaaaacataagctgaacgaaactggttgtcttcccttcccaatccaggacaatctgagaatcccctgcaacattacttaacaaaaaagcaggaataaaattaacaagatgtaacagacataagtcccatcaccgttgtataaagttaactgtgggattgcaaaagcattcaagcctaggcgctgagctgtttgagcatcccggtggcccttgtcgctgcctccgtgtttctccctggatttatttaggtaatatctctcataaatccccgggtagttaacgaaagttaatggagatcagtaacaataactctagggtcattactttggactccctcagtttatccgggggaattgtgtttaagaaaatcccaactcataaagtcaagtaggagattaattcagagctgttgacaattaatcatccggctcgtataatgtgtggaaattgtgagcggataacggaattaggaggttaattaaatgggaaaaaactataaatccctggacagtgtcgtcgcgtctgattttattgcattgggcattaccagtgaagtagcagagaccctgcatgggcgactagctgaaatcgtttgtaattacggagcagcgactccacaaacgtggatcaacatcgcgaatcatatcttaagtccagatctgcctttctccttgcaccagatgttgttttacggatgttataaggattttgggcccgcgcctcctgcttggatcccagaccctgagaaggtaaaaagcaccaacttgggagcattactggagaagcgtggcaaagagttcttaggagtaaagtacaaagacccaatttctagctttagtcactttcaagaatttagtgttcggaatcctgaagtgtattggcgtacagtattaatggatgaaatgaagatcagtttttctaaggacccagaatgtatcctacgtcgagatgatatcaacaatccaggaggtagtgaatggctacctggaggttacttgaacagtgctaagaactgtttaaatgtcaactctaataaaaagttgaacgacactatgatcgtctggcgcgacgaaggcaacgatgatttaccattgaacaaactcacgttagatcagttacggaaacgtgtgtggttagttgggtacgcattagaagagatgggtttggagaaaggttgtgccattgctattgacatgccaatgcacgtcgacgcggtcgttatctatttggctatcgtactagccggatatgtagttgtgtctatcgcggactctttcagtgcccccgagatcagtactcgtctgcgactatccaaggcgaaggctatcttcacgcaggatcacatcattcggggcaaaaaacgaattcctttgtactctcgcgtggttgaggcgaaaagccctatggctatcgtgattccgtgcagcggaagcaatattggtgcagaactacgagatggagacatcagttgggactatttcttagaacgagctaaagagttcaaaaattgtgaattcacagcgcgagaacaaccagtggacgcttatacaaacatcttattttctagtggaacaacaggagaacctaaagcaatcccttggactcaagcgacccctctaaaagctgccgcggatggatggagccatctagacattcgtaagggtgatgtcattgtttggccgacgaatctgggttggatgatgggtccttggctagtttacgcatctctcctaaacggcgccagtatcgctctctacaacgggtctcctctggttagcggattcgcaaaattcgtgcaggacgctaaagtgactatgctaggagtggtcccttctatcgtgcgtagctggaagagcacaaactgcgtctctggatatgattggtctaccatccggtgctttagttcttccggagaagccagcaatgttgatgagtacctgtggttaatgggccgggcaaattacaaaccagttattgagatgtgtggaggaacagaaattgggggagcgttctctgcggggagtttcttgcaagcccaatccctctccagttttagcagtcaatgtatgggctgcactttatacattttggacaagaacggttacccaatgccgaaaaacaaaccgggcattggtgaattagcactaggtccagtaatgttcggagctagtaagacactgttaaatggcaaccatcacgatgtctatttcaaggggatgcccacattaaatggtgaggtcttacgtcgtcacggggacattttcgagttaacctctaatgggtattatcacgctcacgggcgagcggatgacacgatgaacatcggagggattaaaatcagttccatcgaaattgagcgtgtgtgcaatgaggtagacgatcgggtattcgagacaacggccatcggggtgccgcccctcggagggggacccgaacaattggtaattttttttgtcctgaaggattccaacgataccacaatcgacttgaatcagttgcgcctcagcttcaacttaggcttgcagaagaagctaaacccactcttcaaggttacgcgggttgtaccactgtctagcctccctcggactgctacgaataaaatcatgcgccgagtactccgccaacaattcagtcacttcgaataaggaattaggaggttaattaaatgaatcacttgcgagcggaaggtcccgctagtgtactcgctattgggactgccaacccagaaaatattttactccaggatgagttcccggattattacttccgagtcacaaagagcgaacacatgacgcagttaaaagagaagttccgcaaaatctgtgacaagtctatgattcgcaaacgcaattgctttttgaatgaagaacatctgaagcagaatccacgtctggttgagcacgagatgcagactttagacgctcgacaggacatgctagtcgtggaagtcccgaaactgggtaaagacgcgtgtgccaaggccattaaggaatggggtcaacctaagagtaagatcacccatctcatttttaccagtgcgtccacgacagacatgcctggagctgactaccattgtgccaagctcctaggactatctccatctgtgaaacgggtaatgatgtatcagctaggatgttatggtggggggactgtgttacgtatcgcaaaggatatcgcggagaataacaagggggctcgcgtcctagccgtttgctgcgacattatggcgtgcctctttcggggaccctccgagagcgacttggagctattagtaggccaagcgatctttggagatggggccgctgctgttattgttggcgctgaacccgatgagagtgtaggtgagcgcccaattttcgagttggtctccacgggtcagacaattctccccaacagtgaaggcacaattgggggacatatccgggaggcaggactgatctttgacctacataaggacgtcccgatgctcatttctaacaacattgaaaagtgcctgattgaagcgttcaccccaatcggcattagtgattggaatagtatcttctggattactcatcccggaggtaaagccattctagataaggtggaagaaaagttacacttaaagtccgacaagtttgtcgatagtcgtcacgtgctgagcgagcatgggaatatgagtagctctacggttttgttcgttatggacgaattacgaaagcgcagcttggaggagggaaaaagcacgacaggggatggatttgagtggggagttctctttggatttggtcccgggctgacagtagagcgcgtggtggtgcgctccgtgccgattaagtgaggaattaggaggttaattaaatggccgtaaagcacctgattgtattgaaattcaaagatgagatcacggaggcgcagaaggaggagtttttcaagacgtacgtgaacctagtgaatatcatcccggcgatgaaggatgtctattggggtaaagatgtaactcagaaaaacaaggaagaaggttacacccatattgttgaagtcacattcgaaagtgtagagacgatccaagattatattattcatccggctcacgttggatttggagacgtgtatcgttctttttgggagaagttgttaatcttcgactacaccccccgcaaatagggaattaggaggttaattaaatgggcttaagctctgtatgcactttcagtttccaaaccaattatcatacgctcctaaacccccacaataacaacccaaaaacatccttgttgtgctatcgacaccctaaaacgccaatcaagtattcttataacaattttccctccaaacactgctccactaagagcttccacctacaaaacaaatgtagcgaatccttgtccatcgccaagaactccattcgagcagcaaccaccaatcagacagagccacctgagagcgacaatcatagcgtcgcgactaagatcctaaatttcgggaaagcatgctggaaactacaacgaccatacacgatcatcgcgttcaccagttgcgcttgcggtttatttggtaaggaattgctccataataccaacctgatttcctggagtctgatgttcaaagcattttttttcttggtggccatcctatgtatcgcgtcttttacaacaaccatcaatcagatctatgacctccacatcgatcgcattaacaagccagacctcccattagcgtctggtgaaatctctgtcaacaccgcctggattatgagcattattgtagcactgtttgggctaatcattacaatcaagatgaagggtggacccctctacatttttggctattgcttcggaatctttggtggcattgtttacagcgtaccaccgtttcggtggaagcagaatcccagtaccgctttcctattgaactttctggcccacatcatcaccaactttacgttttactacgcaagtcgggcggcactgggcctcccattcgagctgcgacccagttttacgtttctcttagcgttcatgaaaagcatgggaagcgctctcgccctgattaaggatgcctccgacgtggaaggcgacacaaagttcggtatttctacattagcaagcaagtatggttcccgtaacctaacactcttttgttctggaattgtgttactaagttatgtagcagctattctggcaggtatcatttggccccaggccttcaatagcaatgttatgctgttatctcatgcgatcctcgccttctggttaatcctacagacacgggactttgccctcactaattacgatcccgaggcgggccgacgtttttacgagttcatgtggaagctatactatgcagagtacctcgtgtacgtgtttatttaaggaattaggaggttaattaaatgaactgtagcgcatttagtttctggttcgtgtgtaagatcatttttttctttttatcttttcacattcagatttctatcgctaatccgcgcgaaaatttcctcaaatgctttagtaagcacatcccaaacaacgttgcgaatcccaaactggtctacacgcagcacgatcagctctacatgtctatcctgaatagcacaatccagaacttacggttcatctctgatacaacgccaaagcctttagtgattgttacaccgagcaacaattctcatatccaagccacaattttgtgcagtaaaaaggttgggttgcaaatccgaacgcgcagcgggggacacgacgcagagggtatgagttacatttctcaggtccccttcgttgttgtggatctacggaatatgcactccatcaagattgacgtacacagtcagaccgcttgggtcgaagccggagcaaccttaggcgaggtctactattggattaatgagaaaaacgagaacctctctttccctggtggatattgtcctactgtaggtgtcggagggcatttcagtggcggaggctatggggctctcatgcgcaattatggcttggccgcggacaatatcattgacgctcatctcgtgaacgtcgacggtaaggtactcgatcgtaaaagcatgggtgaggatctcttctgggctattcgaggtggtggaggagagaacttcggaattatcgcagcctggaaaattaagttagttgcggtccccagtaaaagcacaatctttagcgtcaaaaagaacatggaaattcatggactcgtaaagctctttaataaatggcagaacattgcatacaaatatgacaaagacctagtgttgatgacccattttattactaaaaatattacggataaccacgggaagaacaagacaacagtacatggttactttagcagcatcttccacggtggggtcgattctctagtagacctgatgaataagtcctttccggaactaggcatcaagaaaactgactgcaaagaattttcctggatcgacacgactatcttctatagtggagtagtaaactttaatacagcaaacttcaaaaaagaaatcctgctagatcgatccgcggggaagaagactgcatttagcattaagctggactatgtaaagaaacccattccggagacagccatggttaaaattttggagaaattgtacgaagaggacgtcggagccggcatgtacgtcctctatccttatggcgggattatggaggaaatcagtgagtccgctatccctttcccccaccgtgcgggtatcatgtacgagttatggtacaccgcgtcctgggaaaagcaggaggacaacgagaaacacatcaactgggtccgttccgtgtacaattttaccaccccttatgtttctcaaaatccgcgactcgcctatttaaactatcgtgacctggacctggggaaaacaaaccacgcgagtcccaataactacacgcaagcacgaatctggggtgaaaagtactttggtaagaatttcaatcgactggttaaagttaagacaaaagtcgatcctaacaatttcttccgaaatgagcaatctattccgcccttgcctcctcatcaccactagggaattaggaggttaattaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtgatttttttaaggcagttattggtgcccttaaacgcctggggatccgctattttgttaattactatttgagctgagtgtaaaataccttacttactcaaaagcattaactaaccataacaatgactaatctctttttttgattgaactccaaactagaatagccatcgagtcagtccatttagttcattattagtgaaagtttgttggcggtgggttatccgttgataaaccaccgtttttgtttgggcaaagtaacgatttgatgcagtgatgggtttaaagataatcccgtttgaggaaatcctgcaggacgacgggaactttaacctgaccgctgctgggttcgtaataattttctaaaattgccgccatggtgcgcccgatcgccaaaccggaaccgttgagagtgtgaacaaattgggtgccttttttgcccttttccttgtagcgaatgttggcccgacgggcttggaaatcgtggaagttagaacaactggaaatttcccggtaggtgttagccgatggtaaccaaacttccaagtcgtagcatttagccgctccaaaacctaaatcaccggtacataattccaccactgagctc

SEQ ID NO: 14 - DNA, 8400 bp, Artificial Sequence
  Description of Artificial Sequence Synthetic polynucleotide
ctcgagaagagtccctgaatatcaaaatggtgggataaaaagctcaaaaaggaaagtaggctgtggttccctaggcaacagtcttccctaccccactggaaactaaaaaaacgagaaaagttcgcaccgaacatcaattgcataattttagccctaaaacataagctgaacgaaactggttgtcttcccttcccaatccaggacaatctgagaatcccctgcaacattacttaacaaaaaagcaggaataaaattaacaagatgtaacagacataagtcccatcaccgttgtataaagttaactgtgggattgcaaaagcattcaagcctaggcgctgagctgtttgagcatcccggtggcccttgtcgctgcctccgtgtttctccctggatttatttaggtaatatctctcataaatccccgggtagttaacgaaagttaatggagatcagtaacaataactctagggtcattactttggactccctcagtttatccgggggaattgtgtttaagaaaatcccaactcataaagtcaagtaggagattaattcagagctgttgacaattaatcatccggctcgtataatgtgtggaaattgtgagcggataacggaattaggaggttaattaaatgggaaaaaactataaatccctggacagtgtcgtcgcgtctgattttattgcattgggcattaccagtgaagtagcagagaccctgcatgggcgactagctgaaatcgtttgtaattacggagcagcgactccacaaacgtggatcaacatcgcgaatcatatcttaagtccagatctgcctttctccttgcaccagatgttgttttacggatgttataaggattttgggcccgcgcctcctgcttggatcccagaccctgagaaggtaaaaagcaccaacttgggagcattactggagaagcgtggcaaagagttcttaggagtaaagtacaaagacccaatttctagctttagtcactttcaagaatttagtgttcggaatcctgaagtgtattggcgtacagtattaatggatgaaatgaagatcagtttttctaaggacccagaatgtatcctacgtcgagatgatatcaacaatccaggaggtagtgaatggctacctggaggttacttgaacagtgctaagaactgtttaaatgtcaactctaataaaaagttgaacgacactatgatcgtctggcgcgacgaaggcaacgatgatttaccattgaacaaactcacgttagatcagttacggaaacgtgtgtggttagttgggtacgcattagaagagatgggtttggagaaaggttgtgccattgctattgacatgccaatgcacgtcgacgcggtcgttatctatttggctatcgtactagccggatatgtagttgtgtctatcgcggactctttcagtgcccccgagatcagtactcgtctgcgactatccaaggcgaaggctatcttcacgcaggatcacatcattcggggcaaaaaacgaattcctttgtactctcgcgtggttgaggcgaaaagccctatggctatcgtgattccgtgcagcggaagcaatattggtgcagaactacgagatggagacatcagttgggactatttcttagaacgagctaaagagttcaaaaattgtgaattcacagcgcgagaacaaccagtggacgcttatacaaacatcttattttctagtggaacaacaggagaacctaaagcaatcccttggactcaagcgacccctctaaaagctgccgcggatggatggagccatctagacattcgtaagggtgatgtcattgtttggccgacgaatctgggttggatgatgggtccttggctagtttacgcatctctcctaaacggcgccagtatcgctctctacaacgggtctcctctggttagcggattcgcaaaattcgtgcaggacgctaaagtgactatgctaggagtggtcccttctatcgtgcgtagctggaagagcacaaactgcgtctctggatatgattggtctaccatccggtgctttagttcttccggagaagccagcaatgttgatgagtacctgtggttaatgggccgggcaaattacaaaccagttattgagatgtgtggaggaacagaaattgggggagcgttctctgcggggagtttcttgcaagcccaatccctctccagttttagcagtcaatgtatgggctgcactttatacattttggacaagaacggttacccaatgccgaaaaacaaaccgggcattggtgaattagcactaggtccagtaatgttcggagctagtaagacactgttaaatggcaaccatcacgatgtctatttcaaggggatgcccacattaaatggtgaggtcttacgtcgtcacggggacattttcgagttaacctctaatgggtattatcacgctcacgggcgagcggatgacacgatgaacatcggagggattaaaatcagttccatcgaaattgagcgtgtgtgcaatgaggtagacgatcgggtattcgagacaacggccatcggggtgccgcccctcggagggggacccgaacaattggtaattttttttgtcctgaaggattccaacgataccacaatcgacttgaatcagttgcgcctcagcttcaacttaggcttgcagaagaagctaaacccactcttcaaggttacgcgggttgtaccactgtctagcctccctcggactgctacgaataaaatcatgcgccgagtactccgccaacaattcagtcacttcgaataaggaattaggaggttaattaaatgaatcacttgcgagcggaaggtcccgctagtgtactcgctattgggactgccaacccagaaaatattttactccaggatgagttcccggattattacttccgagtcacaaagagcgaacacatgacgcagttaaaagagaagttccgcaaaatctgtgacaagtctatgattcgcaaacgcaattgctttttgaatgaagaacatctgaagcagaatccacgtctggttgagcacgagatgcagactttagacgctcgacaggacatgctagtcgtggaagtcccgaaactgggtaaagacgcgtgtgccaaggccattaaggaatggggtcaacctaagagtaagatcacccatctcatttttaccagtgcgtccacgacagacatgcctggagctgactaccattgtgccaagctcctaggactatctccatctgtgaaacgggtaatgatgtatcagctaggatgttatggtggggggactgtgttacgtatcgcaaaggatatcgcggagaataacaagggggctcgcgtcctagccgtttgctgcgacattatggcgtgcctctttcggggaccctccgagagcgacttggagctattagtaggccaagcgatctttggagatggggccgctgctgttattgttggcgctgaacccgatgagagtgtaggtgagcgcccaattttcgagttggtctccacgggtcagacaattctccccaacagtgaaggcacaattgggggacatatccgggaggcaggactgatctttgacctacataaggacgtcccgatgctcatttctaacaacattgaaaagtgcctgattgaagcgttcaccccaatcggcattagtgattggaatagtatcttctggattactcatcccggaggtaaagccattctagataaggtggaagaaaagttacacttaaagtccgacaagtttgtcgatagtcgtcacgtgctgagcgagcatgggaatatgagtagctctacggttttgttcgttatggacgaattacgaaagcgcagcttggaggagggaaaaagcacgacaggggatggatttgagtggggagttctctttggatttggtcccgggctgacagtagagcgcgtggtggtgcgctccgtgccgattaagtgaggaattaggaggttaattaaatggccgtaaagcacctgattgtattgaaattcaaagatgagatcacggaggcgcagaaggaggagtttttcaagacgtacgtgaacctagtgaatatcatcccggcgatgaaggatgtctattggggtaaagatgtaactcagaaaaacaaggaagaaggttacacccatattgttgaagtcacattcgaaagtgtagagacgatccaagattatattattcatccggctcacgttggatttggagacgtgtatcgttctttttgggagaagttgttaatcttcgactacaccccccgcaaatagggaattaggaggttaattaaatgggcttaagctctgtatgcactttcagtttccaaaccaattatcatacgctcctaaacccccacaataacaacccaaaaacatccttgttgtgctatcgacaccctaaaacgccaatcaagtattcttataacaattttccctccaaacactgctccactaagagcttccacctacaaaacaaatgtagcgaatccttgtccatcgccaagaactccattcgagcagcaaccaccaatcagacagagccacctgagagcgacaatcatagcgtcgcgactaagatcctaaatttcgggaaagcatgctggaaactacaacgaccatacacgatcatcgcgttcaccagttgcgcttgcggtttatttggtaaggaattgctccataataccaacctgatttcctggagtctgatgttcaaagcattttttttcttggtggccatcctatgtatcgcgtcttttacaacaaccatcaatcagatctatgacctccacatcgatcgcattaacaagccagacctcccattagcgtctggtgaaatctctgtcaacaccgcctggattatgagcattattgtagcactgtttgggctaatcattacaatcaagatgaagggtggacccctctacatttttggctattgcttcggaatctttggtggcattgtttacagcgtaccaccgtttcggtggaagcagaatcccagtaccgctttcctattgaactttctggcccacatcatcaccaactttacgttttactacgcaagtcgggcggcactgggcctcccattcgagctgcgacccagttttacgtttctcttagcgttcatgaaaagcatgggaagcgctctcgccctgattaaggatgcctccgacgtggaaggcgacacaaagttcggtatttctacattagcaagcaagtatggttcccgtaacctaacactcttttgttctggaattgtgttactaagttatgtagcagctattctggcaggtatcatttggccccaggccttcaatagcaatgttatgctgttatctcatgcgatcctcgccttctggttaatcctacagacacgggactttgccctcactaattacgatcccgaggcgggccgacgtttttacgagttcatgtggaagctatactatgcagagtacctcgtgtacgtgtttatttaaggaattaggaggttaattaaatgaattgtagcacgttcagcttctggttcgtatgtaaaattatcttttttttcctcagttttaatatccaaatctctattgctaacccccaggagaatttcctcaagtgtttcagcgagtacattcctaacaaccctgctccaaaatttatctacacgcaacacgatcaattgtatatgagtgttttaaattccaccatccaaaacttgcgttttacctctgacactacaccaaagcctctcgtcattgtgacgccgagtaatgttagtcatattcaggcgagtattctctgctctaaagttggactccaaatccgcacgcgtagcggcggtcacgatgcggaagggttatcctacattagccaggtgcctttcgctattgttgacttgcgtaatatgcatacagtagtagacattcattcccagacggccgtggaggcaggcgcgacgttgggggaagtttactactggattaatgaaatgaatgaaaatttcagtttccctggaggttactgtccaactgttggagttggaggtcatttttccggaggaggatacggagcgttaatgcggaattacggattagcagcagataatatcatcgacgctcatctagtaaatgtagacggaaaagtattggaccgaaagagtatgggtgaggacttgttctgggctattcgagggggcgggggcgaaaacttcggtatcatcgcagcctgtatcaagctctgggtacccagtaaggccactattttctctgtcaaaaagaacatggagattcacggtctcgtgaagttatttaacaaatggcaaaatattgcctactacgataaagacttgatgttgacgacgcatttccgcacacgcaacattaccgacaaccatgggaataaaacaactgtacacggctatttttctagtatcttcctcgggggcgtagactccctcgtcgatttgatgaataaaagtttcccagaactgggtatcaaaactgactgtaaagaactgtcctggattgataccacgattttctattccggctggtataatacagcctttaagaaagaaattttactggatcgctctgcgggtaaaaagacggctttcagcatcaaactcgactacgttaaaaagctcattccggaaaccgctatggttaaaatcctagagttatacgaagaagaggttggcgtaggcatgtatgtactctacccatacggtggtattatggatgaaatctccgaatccgcaattccatttccccatcgcgcgggtatcatgtatgaactgtatacggcgactgagaaacaggaagacaacgaaaagcacatcaactgggtgcggtccgtctataactttaccaccccttatgtaagtcagaacccgcggctggcatatctaaattatcgggacctggatctaggcaaaacgaaccccgagtctccgaataactatactcaggcgcggatctggggggagaaatactttgggaaaaactttaaccgactcgtaaaggtaaaaaccaaggccgacccgaacaacttcttccgcaacgaacaatctattcccccactccccccacgccatcactagggaattaggaggttaattaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtgatttttttaaggcagttattggtgcccttaaacgcctggggatccgctattttgttaattactatttgagctgagtgtaaaataccttacttactcaaaagcattaactaaccataacaatgactaatctctttttttgattgaactccaaactagaatagccatcgagtcagtccatttagttcattattagtgaaagtttgttggcggtgggttatccgttgataaaccaccgtttttgtttgggcaaagtaacgatttgatgcagtgatgggtttaaagataatcccgtttgaggaaatcctgcaggacgacgggaactttaacctgaccgctgctgggttcgtaataattttctaaaattgccgccatggtgcgcccgatcgccaaaccggaaccgttgagagtgtgaacaaattgggtgccttttttgcccttttccttgtagcgaatgttggcccgacgggcttggaaatcgtggaagttagaacaactggaaatttcccggtaggtgttagccgatggtaaccaaacttccaagtcgtagcatttagccgctccaaaacctaaatcaccggtacataattccaccactgagctc

SEQ ID NO: 15 - DNA, 20 bp, Artificial Sequence
  Description of Artificial Sequence Synthetic oligonucleotide
ggaattaggaggttaattaa

(19) United States
(12) Patent Application Publication
(10) Pub. No.: US 2022/0243236 A1
(43) Pub. Date: Aug. 4, 2022

(54) PRODUCTION OF CANNABINOIDS USING GENETICALLY ENGINEERED PHOTOSYNTHETIC MICROORGANISMS

(71) Applicant: The Regents of the University of California, Oakland, CA (US)

(72) Inventors: Anastasios Melis, El Cerrito, CA (US); Nico Betterle, Pleasanton, CA (US); Diego Alberto Hidalgo Martinez, El Cerrito, CA (US)

(21) Appl. No.: 17/435,695

(22) PCT Filed: Feb. 28, 2020
(86) PCT No.: PCT/US20/20512

§ 371 Date: Sep. 1, 2021

Related U.S. Application Data
(60) Provisional application No. 62/812,906, filed on Mar. 1, 2019.

(51) Int. Cl. C12P 17/06; C12P 7/42; C12N 1/20; C12N 15/74; C12N 15/62; C12N 15/52; C12N 9/00; C12N 9/88; C12N 9/02

(52) U.S. Cl. CPC C12P 17/06; C07K 2319/00; C12N 1/20; C12N 15/74; C12N 15/62; C12N 15/52; C12Y 602/01001; C12Y 121/03007; C12Y 121/03008; C12Y 404/01026; C12N 9/93; C12N 9/88; C12N 9/0004; C12N 2800/101; C12P 7/42

(57) ABSTRACT

The present invention provides methods and compositions for producing cannabinoids in photosynthetic microorganisms, e.g., cyanobacteria.

### CROSS-REFERENCES TO RELATED APPLICATIONS

The present application claims priority to U.S. Provisional Application No. 62/812,906, filed Mar. 1, 2019, the disclosure of which is incorporated herein in its entirety.

### BACKGROUND OF THE INVENTION

Interest in and use of Cannabis sativa products has expanded recently. The specific interaction of cannabinoids with the human endocannabinoid system makes these compounds attractive products to be used for therapeutic purposes and for the treatment of a number of medical conditions. However, understanding of the physicochemical properties and stability of these compounds is limited, production yield is low, and moreover, there is a variable range and mix of products produced by different Cannabis sativa cultivars and other plants. This variability is further exacerbated by variable growth conditions. Agricultural production of cannabinoids is subject to additional challenges such as plant susceptibility to climate and disease, variable yield and product composition due to prevailing cultivation and climatic conditions, the need for extraction of cannabinoids by chemical processing and by necessity, the harvesting of a mix of products that need to be purified and certified for biopharmaceutical use.

The biosynthesis of cannabinoids by engineered microbial strains could be an alternative strategy for the production of these compounds. Accordingly, there is a need to develop the relevant biotechnology and produce the chemically different cannabinoids individually, in pure form, so as to alleviate the above-mentioned difficulties and to enable the unambiguous application of these chemicals in the pharmaceutical industry.

Cannabinoids ate terpenophenolic compounds, generated upon the reaction of a 10-carbon isoprenoid intermediate with a modified fatty acid metabolism precursor as part of the secondary metabolism of Cannabis sativa and other plants (Carvalho et al. (2017) FEMS Yeast Fes 17). More than 100 different chemical species belonging to this class of compounds have been identified (Carvalho et al. (2017), FEMS Yeast Res 17(4); Zirpel et al. (2017), J Biotechn 259, 204-212).

Photosynthetic microorganisms, such as microalgae and cyanobacteria, utilize the methylcrythritol 4-phosphate (MEP) pathway, which generates geranyl diphosphate (GPP) intermediates, and utilize the corresponding isoprenoid pathway enzymes for the biosynthesis of a great variety of endogenously needed terpenoid-type molecules like carotenoids, tocopherols, phytol, sterols, hormones, and many others (see, FIG. 1). The MEP isoprenoid biosynthetic pathway (Lindberg et al. (2010), Metab Eng., 12:70-79) consumes pyruvate and glyceraldehyde-3-phosphate (G3P) as substrates, which are combined to form deoxyxylulose-5-phosphate (DXP), as first described for Escherichia coli (Rohmer et al. (1993). Biochem. J. 295:517-524). DXP is then converted into methylcrythritol phosphate (MEP), which is subsequently modified to form hydroxy-2-methyl-2-butenyl-4-diphosphate (HMBPP). HMBPP is the substrate required for the formation of isopentenyl diphosphate (IPP) and dimethvlallyl diphosphate (DMAPP), which are the universal terpenoid precursors. Cyanobacteria also contain an IPP isomerasc (Ipi in FIG. 1) which catalyzes the inter-conversion of IPP and DMAPP. In addition to reactants G3P and pyruvate, the MEP pathway consumes reducing equivalents and cellular energy in tlie form of NADPH, reduced ferredoxin. CTP, and ATP, ultimately derived from photosynthesis. For reviews, see. e.g., Ershov et al. (2002) J. Bacterial. 184(18):5045-5051: Sharkey et al (2002), Ann. Bot. 101(1):5-18; Bentley et al. (2014), Mol. Plant 7:71-86.

The 5-carbon (5-C) isomeric molecules dimethvlallyl diphosphate (DMAPP) and isopentenyl diphosphate (IPP) are the universal precursors of all isoprenoids (Agranoff et al. (1960); Lichtenthaler (2010)), comprising units of 5-carbon configurations. Two distinct and separate biosynthetic pathways evolved independently in nature to generate these universal DMAPP and IPP precursors (Agranoff et al. (1960), J. Biol. Chem. 236,326-332; Lichtenthaler (2007) Photosynth. Res. 92, 163-179: Lichtenthaler (2010), Chem. Biol. Volatiles, pp 11-47). Most fermentative aerobic and anaerobic bacteria, anoxvgcnic photosynthetic bacteria, cyanobacteria, algae (micro & macro), and chloroplasts in all photosynthetic organisms operate the methylcrythritol 4-phosphate (MEP) pathway, as described above, beginning with glyceraldehyde 3-pltosphatc and pyruvate metabolites (FIG. 1). Archaea, yeast, fungi, insects, animals, and the eukaryotic plant cytosol generally operate the mevalonic acid (MVA) pathway, which begins with acetyl-CoA metabolites (Lichtenthaler (2010) Chem. Biol Volatiles, pp 11-47; McGarvey and Croteau (1995), Plant Cell 7, 1015-1026: Sehwender et al. (2001), Planta 212, 416-423) (FIG. 2). Both pathways result in the synthesis of identical DMAPP and IPP metabolites. Synthesis of geranyl diphosphate (GPP) is due to the presence of a geranyl diphosphate synthase (GPPS) gene that condenses, in a tail to head linear addition, an IPP to a DMAPP molecule (FIG. 3). GPP is the intermediate prenyl metabolite that reacts in the cannabinoid biosynthetic pathway for the synthesis of cannabinoids. Although photosynthetic microorganisms such as microalgae and cyanobacteria utilize the MEP pathway, which generates the DMAPP and IPP precursors, these microorganisms do not need and do not actively and directly express the GPPS enzyme (Bettcrlc and Melis (2018), ACS Synth. Biol. 7, 912-921), nor do they accumulate noticeable levels of the GPP metabolite.

The dedicated pathway for the cellular synthesis of cannabinoids (FIG. 5) commences with hexanoic acid, a 6-carbon intermediate in the fatty acid biosynthetic pathway. Action by acyl activating enzyme 1 (AEE1) converts the hexaooid acid to its coenzyme A (Hexanoyl-CoA) form (Stout et al. (2012), Plant J 71:353-65; Carvalho et al. (2017), FEMS Yeast Res 17; Zirpel et al. (2017), J Biotechn 259, 204-212). Action of the enzymes olivetol synthase (OLS), which is a type III polyketide synthase; and olivetolic acid cyclase (OAC), which is a polyketide cyclase, combines one molecule of hexanoyl-CoA and three molecules of malonyl-CoA reactants, followed by cyclization of the C2-C7 aldol portion of the molecule to generate olivetolic acid, a 12-carbon pathway (C₁₂H₁₆O₄) intermediate (Gagne et al. (2012); Rahatjo et al. (2004)). A geranyl diphosphate olivetolic acid prenyl transferase, cannabigeroiic acid synthase (CBGAS), catalyzes the C-alkylation of olivetolic acid by geranyl diphosphate (GPP) to form cannabigeroiic acid (CBGA), a 12-carbon (C₂₂H32O₄) cannabinoid intermediate (Fellermeier and Zenk 1998). Subsequent catalysis by the cannabidiolic acid synthase (CBDAS) results in the oxidative cyclization of the monoterpene portion of the CBGA, leading to the formation of cannabidiolic acid (CBDA), a 12-carbon (C₂₂H₃₀₄) oxidized derivative of cannabigeroiic acid (Morimoto et al. (1998). Phytochemistry 49:1525-1529; Sirikantaramas et al. (2004), J Biol Chem 279:39767-39774: Taura et al. (2007), FEBS Lett 581:2929-2934). A decarboxylated and biologically active but non-psychoactive form of the latter (cannabidiol) typically occurs by a non-enzymatic process that may happen during heating or exposure to sunlight (de Meijer et al., Genetics 163,335-346, 2003).

Alternative oxidocyclase enzymes catalyze the oxidative cyclization of the monoterpene moiety of CBGA for the biosynthesis of Δ9-tetrahydrocannanbinolic acid (Δ9-THCA) and cannabichromenic acid (CBCA) (Morimoto et al. (1998), Phytochemistry 49:1525-1529; Sirikantaramas et al. (2004), J Biol Chem 279:39767-39774; Taura et al. (2007), FEBS Lett 581:2929-2934). The latter are chemical isomers of the CBDA, having the same C₂₂H₃₀O₄ chemical formula. Decarboxylated and biologically active (psychoactive) forms of the Δ9-THCA and CBCA cannabinoids (Δ9-THC and CBC, respectively) typically occur by a non-enzymatic process that may happen during heating or exposure to sunlight (de Meijer et al. (2003), Genetics 163,335-346).

The present invention provides improved methods and compositions for producing cannabinoids in photosynthetic microorganisms, allowing the production of highly pure cannabinoids that can bo used in numerous biotechnological, pharmaceutic, and cosmetics applications.

### BRIEF SUMMARY OF THE INVENTION

The current invention provides new methods for generating purified cannabinoids, e.g., cannabidiolic acid, in photosvnthetic microorganisms, e.g. cyanobacteria and microalgae. The cannabidiolic acid (CBDA) and other cannabinoids produced using the present methods are derived via photosynthesis from sunlight, carbon dioxide, and water.

The invention takes advantage of improvements in the engineering of photosynthetic microorganisms, e.g., cyanobacteria, which, upon suitable genetic modification, can be used to produce large quantities of highly pure cannabinoids such as cannabidiolic acid. The invention provides methods and compositions for generating and harvesting cannabidiolic acid and other cannabinoids from genetically modified cyanobacteria or other photosynthetic microorganisms. Such genetically modified microorganisms can be used commercially in an enclosed mass culture system, e.g., a photobioreactor, to provide a source of highly pure and valuable compounds for use in various industries, such as the medical, pharmaceutical, and cosmetics industries.

In one aspect, the present disclosure provides a method for producing cannabinoids in a photosynthetic microorganism, the method comprising (i) introducing into the microorganism: a polynucleotide encoding a GPPS polypeptide; and one or more polynucleotides encoding AAE1, OLS, OAC, CBGAS polypeptides and an oxidocyclase selected from the group consisting of CBDAS, THCAS, and CBCAS; wherein the polynucleotide encoding the GPPS polypeptide is operably linked to a first promoter, and the one or more polynucleotides encoding the AAE1, OLS, OAC, CBGAS polypeptides and the oxidocyclase are operably linked to one or more additional promoters; and (ii) culturing the microorganism under conditions in which the GPPS, AAE1, OLS, OAC, CBGAS polypeptides and the oxidocyclase are expressed and wherein cannabinoid biosynthesis takes place.

In some embodiments, the photosynthetic microorganism modified in accordance with the disclosure is cyanobacteria. In some embodiments, the GPPS polypeptide is a fusion protein encoded by a polynucleotide encoding GPPS fused to the 3′ end of a leader nucleic acid sequence encoding a protein that is expressed in cyanobacteria at a level of at least 1% of the total cellular protein. In some embodiments, the GPPS polypeptide is an nptI*GPPS fusion protein. In some embodiments, the GPPS polypeptide comprises an amino acid sequence that is at least 90% or 95% identical to SEQ ID NO:2. In some embodiments, the GPPS polypeptide comprises the amino acid sequence of SEQ ID NO:2. In some embodiments, the polynucleotide encoding the GPPS polypeptide comprises a nucleotide sequence that is at least 90% or 95% identical to SEQ ID NO:1. In some embodiments, the polynucleotide encoding the GPPS polypeptide comprises the nucleotide sequence of SEQ ID NO:1.

In some embodiments, the AAE1 polypeptide used in accordance with the disclosure comprises an amino acid sequence that is at least 90% or 95% identical to SEQ ID NO:4. In some embodiments, the AAE1 polypeptide comprises the amino acid sequence of SEQ ID NO:4. In some embodiments, the polynucleotide encoding the AAE1 polypeptide comprises a nucleotide sequence that is at least 90% or 95% identical to nucleotides 636-2798 of SEQ ID NO:3. In some embodiments, the polynucleotide encoding the AAE1 polypeptide comprises nucleotides 636-2798 of SEQ ID NO:3. In some embodiments, the OLS polypeptide used in accordance with the disclosure comprises ait amino acid sequence that is at least 90% or 95% identical to SEQ ID NO:5. In some embodiments, the OLS polypeptide comprises the amino acid sequence of SEQ ID NO:5. In some embodiments, the polynucleotide encoding the OLS polypeptide comprises a nucleotide sequence that is at least 90% or 95% identical to nucleotides 2819-3973 of SEQ ID NO:3. In some embodiments, the polynucleotide encoding the OLS polypeptide comprises nucleotides 2819-3973 of SEQ ID NO:3.

In some embodiments, the OAC polypeptide used in accordance with the disclosure comprises an amino acid sequence that is at least 90% or 95% identical to SEQ ID NO:6. In some embodiments, the OAC polypeptide comprises the amino acid sequence of SEQ ID NO:6. In some embodiments, the polynucleotide encoding the OAC polypeptide comprises a nucleotide sequence that is at least 90% or 95% identical to nucleotides 3994-4299 of SEQ ID NO:3. In some embodiments, the polynucleotide encoding the OAC polypeptide comprises nucleotides 3994-4299 of SEQ ID NO:3. In some embodiments, the CBGAS polypeptide used in accordance with the disclosure comprises an amino acid sequence that is at least 90% or 95% identical to SEQ ID NO:7. In some embodiments, the CBGAS polypeptide comprises the amino acid sequence of SEQ ID NO:7. In some embodiments, the polynucleotide encoding the CBGAS polypeptide comprises a nucleotide sequence that is at least 90% or 95% identical to nucleotides 4320-5507 of SEQ ID NO:3. In some embodiments, the polynucleotide encoding the CBGAS polypeptide comprises nucleotides 4320-5507 of SEQ ID NO:3.

In some embodiments, the oxidocvclase used in accordance with the disclosure is CBDAS, and the CBDAS comprises an amino acid sequence that is at least 90% or 95% identical to SEQ ID NO:8. In some embodiments, the oxidocyclase is CBDAS, and the CBDAS comprises the amino acid sequence of SEQ ID NO:8. In some embodiments, the polynucleotide encoding the CBDAS comprises a nucleotide sequence that is at least 90% or 95% identical to nucleotides 5528-7162 of SEQ ID NO:3. In some embodiments, the polynucleotide encoding the CBDAS comprises nucleotides 5528-7162 of SEQ ID NO:3. In some embodiments, the oxidocyclase used in accordance with the disclosure is THCAS, and the THCAS comprises an amino acid sequence that is at least 90% or 95% identical to SEQ ID NO:10. In some embodiments, the oxidocyclase is THCAS, and the THCAS comprises the amino acid sequence of SEQ ID NO:10. In some embodiments, the polynucleotide encoding the THCAS comprises a nucleotide sequence that is at least 90% or 95% identical to SEQ ID NO:9. In some embodiments, the polynucleotide encoding the THCAS comprises the nucleotide sequence of SEQ ID NO:9.

In some embodiments, the oxidocyclase used in accordance with the disclosure is CBCAS, and the CBCAS comprises an amino acid sequence that is at least 90% or 95% identical to SEQ ID NO:12. In some embodiments, the oxidocyclase is CBCAS, and the CBCAS comprises the amino acid sequence of SEQ ID NO:12. In some embodiments, the polynucleotide encoding the CBCAS comprises a nucleotide sequence that is at least 90% or 95% identical to SEQ ID NO:11. In some embodiments, the polynucleotide encoding the CBCAS comprises the nucleotide sequence of SEQ ID NO:11.

In some embodiments, two or more of the polynucleotides encoding the A AE1, OLS, OAC, CBGAS polypeptides and the oxidocyclase are present within a single operon. In some embodiments, all of the polynucleotides encoding the AAE1, OLS, OAC, CBGAS polypeptides and the oxidocyclase are present within a single operon. In some embodiments, the operon is at least 90% or 95% identical to SEQ ID NO:3, SEQ ID NO:13, or SEQ ID NO:14. In some embodiments, the operon comprises SEQ ID NO:3, SEQ ID NO:13, or SEQ ID NO:14. In some embodiments, the first and or additional promoters used in accordance with the disclosure are selected from the group consisting of a cpc promoter, a psbA2 promoter, a glgA1 promoter, a Ptrc promoter, and a 17 promoter.

In some embodiments, one or more of the polynucleotides encoding the GPPS, AAE1, OLS, OAC, CBGAS polypeptides and the oxidocyclase are codon optimized for the photosynthetic microorganism. In some embodiments, the microorganism modified in accordance with the disclosure is from a genus selected from the group consisting of Synechocystis, Synechococcus, Athrospira, Nostoc, and Anabaena. In some embodiments, one or more of the coding sequences for the GPPS, AAE1, OLS, OAC, CBGAS polypeptides and the oxidocyclase are preceded by a ggaattaggaggttaattaa ribosome binding site (RBS).

In some embodiments, the method further comprises a step (c) comprising isolating cannabinoids from the microorganism or from the culture medium. In some embodiments, the cannabinoids are isolated from the surface of the liquid culture as floater molecules. In some embodiments, the cannabinoids are extracted from the interior of the microorganism. In some embodiments, the cannabinoids are extracted from a disintegrated cell suspension produced by isolating the microorganism and disintegrating it by forcing it through a French press, subjecting it to sonication, or treating it with glass beads. In some embodiments, the disintegrated cell suspension is supplemented with H₂SO₄ and 30% (w:v) NaCl at a volume-to-volume ratio of (cell suspension/H₂SO₄/NaCl=3/0.12/0.5). In some embodiments, the cannabinoids are extracted from the H₂SO₄ and NaCl-treated disintegrated cell suspension upon incubation with an organic solvent. In some embodiments, the organic solvent is hexane or heptane. In some embodiments, the organic solvent is ethyl acetate, acetone, methanol, ethanol, or propanol. In some embodiments, the microorganism is freeze-dried. In some embodiments, the cannabinoids are extracted from the freeze-dried microorganism with an organic solvent. In some embodiments, the organic solvent is methanol, acctonitrile, ethyl acetate, acetone, ethanol, propanol, hexane, or heptane. In some embodiments, the organic solvent is dried by solvent evaporation, leaving the cannabinoids in pure form.

In another aspect, the present disclosure provides a photosynthetic microorganism produced using any of the methods described herein. In another aspect, the present disclosure provides a photosynthetic microorganism comprising: (i) a polynucleotide encoding a GPPS polypeptide; and (ii) one or more polynucleotides encoding AAE1, OLS, OAC, CBGAS polypeptides and an oxidocyclase selected from the group consisting of CBDAS, THCAS, and CBCAS: wherein the polynucleotide encoding the GPPS polypeptide is operably linked to a first promoter, and wherein the one or more polynucleotides encoding the AAE1, OLS, OAC, CBGAS polypeptides and the oxidocyclase are operably linked to one or more additional promoters.

In some embodiments, the photosynthetic microorganism is cyanobacteria. In some embodiments, the GPPS polypeptide is a fusion protein encoded by a polynucleotide encoding GPPS fused to the 3′ end of a leader nucleic acid sequence encoding a protein that is expressed in cyanobacteria at a level of at least 1% of the total cellular protein. In some embodiments, the GPPS polypeptide is an nptI*GPPS fusion protein. In some embodiments, the GPPS polypeptide comprises an amino acid sequence tltat is at least 90% or 95% identical to SEQ ID NO:2. In some embodiments, the GPPS polypeptide comprises the amino acid sequence of SEQ ID NO:2. In some embodiments, the polynucleotide encoding the GPPS polypeptide comprises a nucleotide sequence that is at least 90% or 95% identical to SEQ ID NO:1. In some embodiments, the polynucleotide encoding the GPPS polypeptide comprises the nucleotide sequence of SEQ ID NO:1.

In some embodiments, the AAE1 polypeptide comprises an amino acid sequence that is at least 90% or 95% identical to SEQ ID NO:4. In some embodiments, the AAE1 polypeptide comprises the amino acid sequence of SEQ ID NO:4. In some embodiments, the polynucleotide encoding the AAE i polypeptide comprises a nucleotide sequence that is at least 90% or 95% identical to nucleotides 636-2798 of SEQ ID NO:3. In some embodiments, the polynucleotide encoding the AAE1 polypeptide comprises nucleotides 636-2798 of SEQ ID NO:3. In some embodiments, the OLS polypeptide comprises an amino acid sequence that is at least 90% or 95% identical to SEQ ID NO:5. In some embodiments, the OLS polypeptide comprises the amino acid sequence of SEQ ID NO:5. In some embodiments, the polynucleotide encoding the OLS polypeptide comprises a nucleotide sequence that is at least 90% or 95% identical to nucleotides 2819-3973 of SEQ ID NO:3. In some embodiments, the polynucleotide encoding the OLS polypeptide comprises nucleotides 2819-3973 of SEQ ID NO:3.

In some embodiments, the OAC polypeptide comprises an amino acid sequence that is at least 90% or 95% identical to SEQ ID NO:6. In some embodiments, the OAC polypeptide comprises the amino acid sequence of SEQ ID NO:6. In some embodiments, the polynucleotide encoding the OAC polypeptide comprises a nucleotide sequence that is at least 90% or 95% identical to nucleotides 3994-4299 of SEQ ID NO:3. In some embodiments, the polynucleotide encoding the OAC polypeptide comprises nucleotides 3994-4299 of SEQ ID NO:3. In some embodiments, the CBGAS polypeptide comprises an amino acid sequence that is at least 90% or 95% identical to SEQ ID NO:7. In some embodiments, the CBGAS polypeptide comprises the amino acid sequence of SEQ ID NO:7. In some embodiments, the polynucleotide encoding the CBGAS polypeptide comprises a nucleotide sequence that is at least 90% or 95% identical to nucleotides 4320-5507 of SEQ ID NO:3. In some embodiments, the polynucleotide encoding the CBGAS polypeptide comprises nucleotides 4320-5507 of SEQ ID NO:3.

In some embodiments, the oxidocyclase is CBDAS, and the CBDAS comprises an amino acid sequence that is at least 90% or 95% identical to SEQ ID NO:8. In some embodiments, the oxidocyclase is CBDAS, and the CBDAS comprises the amino acid sequence of SEQ ID NO:8. In some embodiments, the polynucleotide encoding the CBDAS comprises a nucleotide sequence that is at least 90% or 95% identical to nucleotides 5528-7162 of SEQ ID NO:3. In some embodiments, the polynucleotide encoding the CBDAS comprises nucleotides 5528-7162 of SEQ ID NO:3. In some embodiments, the oxidocyclase is THCAS, and the THCAS comprises an amino acid sequence that is at least 90% or 95% identical to SEQ ID NO:10. In some embodiments, the oxidocyclase is THCAS, and the THCAS comprises the amino acid sequence of SEQ ID NO:10. In some embodiments, the polynucleotide encoding the THCAS comprises a nucleotide sequence that is at least 90% or 95% identical to SEQ ID NO:9. In some embodiments, the polynucleotide encoding the THCAS comprises the nucleotide sequence of SEQ ID NO:9.

In some embodiments, the oxidocyclase is CBCAS, and the CBCAS comprises an amino acid sequence that is at least 90% or 95% identical to SEQ ID NO:12. In some embodiments, the oxidocyclase is CBCAS, and the CBCAS comprises the amino acid sequence of SEQ ID NO:12. In some embodiments, the polynucleotide encoding the CBCAS comprises a nucleotide sequence that is at least 90% or 95% identical to SEQ ID NO:11. In some embodiments, the polynucleotide encoding the CBCAS comprises the nucleotide sequence of SEQ ID NO:11.

In some embodiments, two or more of the polynucleotides encoding the AAE1, OLS, OAC, CBGAS polypeptides and the oxidocyclase are present within a single operon. In some embodiments, all of the polynucleorides encoding the AAE1, OLS, OAC, CBGAS polypeptides and the oxidocyclase are present within a single operon. In some embodiments, the operon is at least 90% or 95% identical to SEQ ID NO:3, SEQ ID NO:13, or SEQ ID NO:14. In some embodiments, the operon comprises SEQ ID NO:3, SEQ ID NO:13, or SEQ ID NO:14. In some embodiments, the first and or additional promoters are selected from the group consisting of a cpe promoter, a psbA2 promoter, a glgAl promoter, a Ptrc promoter, and a T7 promoter.

In some embodiments, one or more of the polynucleotides encoding the GPPS, AAE1, OLS, OAC, CBGAS polypeptides and the oxidocyclase are codon optimized for the photosynthetic microorganism. In some embodiments, the microorganism is from a genus selected from the group consisting of Synechocystis, Synechococcus, Athrospira, Nostoc, and Anabaena. In some embodiments, one or more of the coding sequences for the GPPS, AAE1, OLS, OAC, CBGAS polypeptides and the oxidocyclase are preceded by a ggaattaggaggnaattaa ribosome binding site (RBS).

In other aspects, the present disclosure provides a polynucleotide encoding a GPPS, AAE1, OLS, OAC, CBGAS, CBDAS, THCAS polypeptide and or CBCAS polypeptide, wherein the polynucleotide is codon optimized for cyanobacteria or other photosynthetic microorganism. In some embodiments, the polynucleotide is at least 90% or 95% identical to a sequence selected from the group consisting of SEQ ID NO:1, SEQ ID NO:3, SEQ ID NO:9, SEQ ID NO:11, SEQ ID NO:13, SEQ ID NO:14, nucleotides 636-2798 of SEQ ID NO:3, nucleotides 2819-3973 of SEQ ID NO:3, nucleotides 3994-4299 of SEQ ID NO:3, nucleotides 4320-5507 of SEQ ID NO:3, and nucleotides 5528-7162 of SEQ ID NO:3. In some embodiments, the polynucleotide comprises a sequence selected from the group consisting of SEQ ID NO:1, SEQ ID NO:3, SEQ ID NO:9, SEQ ID NO:11, SEQ ID NO:13, SEQ ID NO:14, nucleotides 636-2798 of SEQ ID NO:3, nucleotides 2819-3973 of SEQ ID NO:3, nucleotides 3994-4299 of SEQ ID NO:3, nucleotides 4320-5507 of SEQ ID NO:3, and nucleotides 5528-7162 of SEQ ID NO:3.

In another aspect, the present disclosure provides an expression cassette comprising any of the herein-described polynucleotides. In another aspect, the present disclosure provides a host cell comprising any of the herein-described polynucleotides or expression cassettes. In another aspect, the present disclosure provides a cell culture comprising any of the herein-described microorganisms or host cells.

In another aspect, the present disclosure provides a method for producing cannabinoids, the method comprising culturing any of the herein-described photosynthetic microorganisms or host cells under conditions in which the GPPS, AAE1, OLS, OAC, CBGAS polypeptides and the aoxidocyclase are expressed and wherein cannabinoid biosynthesis takes place.

In some embodiments, the method further comprises a step (c) comprising isolating cannabinoids from the microorganism or from the culture medium. In some embodiments, the cannabinoids are isolated from the surface of the liquid culture as floater molecules. In some embodiments, the cannabinoids are extracted from the interior of the microorganism. In some embodiments, the cannabinoids ate extracted from a disintegrated cell suspension produced by isolating the microotganism and disintegrating it by forcing it through a French press, subjecting it to sonication, or treating it with glass heads. In some embodiments, the disintegrated cell suspension is supplemented with H₂SO₄ and 30% (w:v) NaCl at a volume-to-volume ratio of (cell suspension/H₂SO₄/NaCl=3/0.12/0.5). In some embodiments, the cannabinoids are extracted from the H₂SO₄ and NaCl-treated disintegrated cell suspension upon incubation with an organic solvent. In some embodiments, the organic solvent is hexane or heptane. In some embodiments, the organic solvent is ethyl acetate, acetone, methanol, ethanol, or propanol. In some embodiments, the microorganism is freeze-dried. In some embodiments, the cannabinoids are extracted from the freeze-dried microorganism with an organic solvent. In some embodiments, the organic solvent is methanol, acetonitrile, ethyl acetate, acetone, ethanol, propanol, hexane, or heptane. In some embodiments, the organic solvent is dried by solvent evaporation, leaving the cannabinoids in pure form.

### BRIEF DESCRIPTION OF THE DRAWINGS

FIG. 1. Terpenoid biosynthesis via the endogenous MEP (methylerythritol-4-phosphate) pathway in photosynthetic microorganisms, e.g. Synechocystis sp. Abbreviations used: G3P, glyceraldehyde 3-phosphate: Dxs, deoxyxylulose 5-phosphate synthase: Dxr, deoxyxylulose 5-phosphate reductoisomerase; IspD, diphosphocytidylyl methylcrythritol synthase; IspE, diphosphocytidylyl methylerythritol kinase; IspF, methyl crythritol-2,4-cyclodiphosphate synthase; IspG, hydroxymethylbutenyl diphosphate synthase; IspH, hydroxymethylbutenyl diphosphate reductase; Ipi, IPP isomerase.

FIG. 2. Terpenoid biosynthesis via the heterologous MVA (mevalonic acid) pathway in photosynthetic microorganisms, e.g. Synechocystis sp. Abbreviations used: AtoB, acetyl-CoA acetyl transferase; HmgS, Hmg-CoA synthase: HmgR, Hmg-CoA reductase; MK, mevalonic acid kinase; PMK, mevalonic acid 5-phosphate kinase; PMD, mevalonic acid 5-diphoshate decarboxylase: Fni, IPP isomerase.

FIG. 3. Biosynthesis of geranyl diphosphate (GPP) by the action of the enzyme genmyl diphosphate synthase (GPPS). GPP is the first precursor to mono-, sesqui-, di-, tri-, tetra-terpenoids and all their derivatives.

FIG. 4. Protein expression analysis of Synechocystis wild type (WT) and transformant strains. Total cell proteins were resolved by SDS-PAGE, transferred to nitrocellulose and probed with specific α-GPPS2 polyclonal antibodies. Individual native and heterologous proteins of interest are indicated on the right side of the blot. Transformant lines expressing GPPS along with SmR (GPPS-SmR) or the fusion NptI*GPPS only (NptI*GPPS) were loaded onto the gel. Sample loading corresponds to 0.125 μg of chlorophyll for the Western blot analysis. Upper arrow shows the presence of the NptI*GPPS fusion protein. Upper arrow shows a strong specific cross-reaction the polyclonal Picea abies GPPS2 antibodies and a protein band migrating to 62 kD in the Npti*GPPS2 fusion transformant, showing that the P_{TRC}-Nptl*GPPS construct was truly overexpressed at the protein level in Synechocystis. Lower arrow shows a faint cross-reaction at ˜32 kD observed in wild type and transformants. By reference to the Mycoplasma tuberculosis GPPS, GenBank accession number AF082325.1, this was assigned to ORF slr0611 encoding a putative prenyltransferase of 32 kD, which could thus account for the low-level expression of the native GPPS in Synechocystis.

FIG. 5. The cannabinoid biosynthesis pathway in photosynthetic microorganisms, e.g. Synechocystis sp. Abbreviations used: AAE1, Acyl Activating Enzyme 1: OLS, Olivetol synthase; OAC, Olivetolic acid Cyclase; CBGAS, Cannabigerolic acid syntliase; CBDAS, Cannabidiolic acid synthase.

FIG. 6. Gas chromatography detection with a flame ionization detector (GC-FID) of floater extracts from Synechocystis wild type (WT) untreated and cultures treated with cannabidioi (CBD). (Upper panel) GC-FID analysis of heptane extracts from a Synechocystis wild type untreated culture. Floater extracts from wild type cultures displayed a flat profile, without any discernible peaks. (Lower panel) GC-FID analysis of floater extracts from a Synechocystis culture incubated in the presence of cannabidiol. Cannabidiol was the major product detected, showhng a retention time of 9.2 min under these experimental conditions. Smaller amounts of an additional compound with retention times of 10.3 min were also detected as secondary product of the process (See, e.g., Dussy F E et al. (2005), Isolation of D9-THCA-A from hemp and analytical aspects concerning the determination of D9-THC in cannabis products, Forensic Science International 149:3-10; Ibrahim E A et al. (2018) Determination of acid and neutral cannabinoids in extracts of different strains of Cannabis sativa using GC-FID. Planta Med 84:250-259).

FIG. 7. Spectrophotometric detection of cannubidiolic acid and cannabidiol in heptane solution. (Upper panel) Absorbance spectrum of cannubidiolic acid (CBDA) showing UV maxima at 225 and 270 nm from which the concentration of CBDA can be calculated, (lower panel) Absorbance spectrum of cannabidiol (CBD) showing a UV peak at 214 nm and a shoulder at 233 nm from which the concentration of CBD can be calculated. A system of equations based on the extinction coefficients of CBDA and CBD at the above-mentioned wavelengths permits delineation of the concentration of the two cannabinoids in a mix solution. Cannabinoids can be siphoned off the top of the liquid medium from transformant Synechocystis cultures after applying a known volume of heptane solvent as over-layer (see, e.g., U.S. Pat. No. 9,951,354).

FIGS. 8A-8B. Linear addition of Synechocystis CBDA transforming constructs. FIG. 8A: Map of the upper (construct L#1: 5,300 nt) and lower (construct L#2: 4,640 nt) Synechocystis codon-optimized cannabidiolic acid biosynthetic pathway-encoding genes. L#1 harbored the AAE1, OLS, CMC, and zeocin (zeoR) resistance genes. L#2 harbored the OLS, OAC, CBGAS, CBDAS, and chloramphenicol (cmR) encoding genes. Synechocystis was transformed linearly (sequentially) first with construct L#1 and, upon reaching homoplasmy, with L#2. FIG. 8B: Genomic DNA PCR analysis testing for the insertion of the CBDA-related genes in Synechocystis transformants. Primers <OLS for> and <cmR rev> were employed for screening the transformants harboring the genes required for CBDA synthesis in Synechocystis. Genomic DNA from wild-type (WT) and the L#1 transformant strains, with the latter harboring only the upper CBDA-encoding genes, were used as controls. Both wild type and L#1 PCR products generated unspecific 700 bp size products, whereas four different cell lines (O19, N13, N15, and N17), comprising both the L#1 and L#2 constructs, generated the expected 3,822 bp size product. These results showed the full integration of the CBDA biosynthetic pathway in Synechocystis.

FIGS. 9A-9B. Linear addition of Synechocystis CBDA transforming constructs. FIG. 9A: Map of the upper (construct L#2; 5300 nt) and lower (construct L#2: 4640 nt) Synechocystis codon-optimized cannabidiolic acid (CBDA) biosynthetic pathway-encoding genes. L#1 harbored the AAE1, OLS, OAC and zeocin resistance cassette genes. L#2 harbored the OLS, OAC, CBGAS, CBDAS, and cmR encoding genes. Synechocystis was transformed linearly (sequentially) with construct L#1 and, upon reaching homoplasmy, with L#2. FIG. 9B: Genomic DNA PCR analysis testing for the correct insertion of individual CBDA biosynthesis-related genes in Synechocystis transformants. (Upper left panel) Primers <OLS for> and <cpc-ds rev> generated a 1,978 bp product in the L#1 transformant and 5,130 bp products in three different transformants comprising both the L#1 and L#2 constructs. PCR using WT genomic DNA did not generate a PCR product, as expected. (Upper right panel) Primers <OACfor> and <vpc-ds rev> generated a 1,202 bp product in the L#1 transfonnant and 4,354 bp products in three different transformants comprising both the L#1 and L#2 constructs. PCR using WT genomic DNA did not generate a PCR product, as expected. (Lower left panel) Primers <cpc-us for> and <OAC rev> generated 4,320 bp products both in the Ltf 1 transformant and in three different transformants comprising the L#1 and L#2 constructs. PCR using WT genomic DNA did not generate a PCR product, as expected. (Lower right panel) Primers <cpc-us for> and <OLS rev> generated 3,542 bp product both in the L#1 transformant and in three different transformants comprising the L#1 and L#2 constructs. PCR using WT genomic DNA did not generate a PCR product, as expected. These results strengthened the notion of correct insertion of the entire heterologous CBDA biosynthetic pathway genes in Synechocystis.

FIGS. 10A-10B. Linear addition of Synechocystis CBDA transforming constructs. FIG. 10A (upper): Map of CBDA biosynthetic pathway encoding genes installed as an operon in the genomic DNA of Synechocystis. Transgenic operon replaced the native cpc operon, under the control of the P_{TRC} promoter. FIG. 10A (lower): Map of the heterologous mevalonic acid pathway-encoding genes installed in the Synechocystis glgA1 locus, expressed under the control of the P_{TRC} promoter. FIG. 10B: RT-PCR analysis of Synechocystis CBDA transformants offers evidence of transcription and mRNA accumulation of the cell endogenous 16 rRNA gene (200 bp product), as well as the heterologous AAE1 transgene (275 bp product), CBDAS transgene (295 bp product), and GPPS transgene (286 bp product). These results validate the successful installation and expression of two exogenous operons, shown in FIG. 10A, comprising twelve heterologous transgenes expressed in Synechocystis.

FIGS. 11A-11C. Parallel addition of Synechocystis CBDA transforming constructs. FIG. 11A: Map of the CBDA construct P#1 (6,674 nt) in the cpc operon locus harboring the AAE1, OLS, OAC, atoB, cmR genes, and CBDA construct P#2 (6,573 nt) in the psbA2 gene locus of Synechocystis harboring the nptI*GPPS fusion, CBCAS, CBDAS, and smR encoding genes. FIG. 11B: Screening by PC R analysis of a set of colonies transformed with CBDA construct P#1. For verification of insertion <cps-us for> and <cpc-ds rev> primers were used. Colonics 8, 9, 17 and 20 showed the expected size products. FIG. 11C: Screening by PCR analysis of the second set of colonies transformed with CBDA construct P#1. For verification of correct insertion, <cpc-usfor> and <AAE1 rev> printers were used. Again, colonies 8, 9, 17 and 20 showed the right size products. The results showed that colonies 8, 9, 17 and 20 are successful CBDA construct P#1 transformants.

FIGS. 12A-12B. Parallel addition of Synechocystis CBDA transforming constructs. FIG. 12 A: Map of the CBDA construct P#1 (6,674 nt) in the cpc operon locus liarboring the AAE1, OLS, OAC, atoB, cmR genes, and CBDA construct P#2 (6,573 nt) in the psbA2 gene locus of Synechocystis harboring the nptI*GPPS fusion, CBGAS, CBDAS, and smR encoding genes. FIG. 12B: Screening by PCR analysis of a set of colonies transformed with CBDA construct P#2. For verification of correct insertion, straias were tested with primers <psbA2-us for> and <psbA2-ds rev> (CBDAS) (left side of the construct map and gel panel), spanning the full length of the insert. Also. <CBDAS for> and <psbA2-ds rev> primers were used (right side of the construct map and gel panel) to test for the location of the CBDAS gene in relation to the psbA2 DS gene region. Colonies 1, 2, 4, 5, 6 and 7 had the correct product size and insertion position in the psbA2 gene locus, showing successfully transformation of these heterologous genes.

FIG. 13. SDS-PAGE (left panel) and Western blot analysis (right panel) of wild type and three CBDA biosynthetic pathway transformants, as described in FIG. 12. Lane WT: wild type. Lanes 4, 5, 6: Same as lanes 4, 5, and 6 in FIG. 12. Wild type and transformant cells were grown under the same experimental conditions. Lanes were loaded with 0.3 μg cellular chlorophyll. The Coomassie stain in the SDS-PAGE panel showed the distinct presence of the NptI*GPPS fusion plus CBDAS proteins, both migrating in the vicinity of 62 kD, and the presence of the CBGAS protein migrating to about 45 kD. Polyclonal antibodies against the GPPS protein were used to show the presence of the NptI*GPPS fusion protein. Only transformants in lanes 4, 5, and 6 were positive in the SDS-PAGE and Western blot analysis for the expected NptI*GPPS, CBDAS, and CBGAS proteins.

FIG. 14. Cyanobacterial cannabinoid analysis by GC-MS. FIG. 14A: standards; FIG. 14B; cell extracts.

FIG. 15. Codon-optimized DNA sequences in operon configuration of the cannabinoid biosynthesis pathway shown in FIG. 5, leading to the synthesis of cannabidiolic acid.

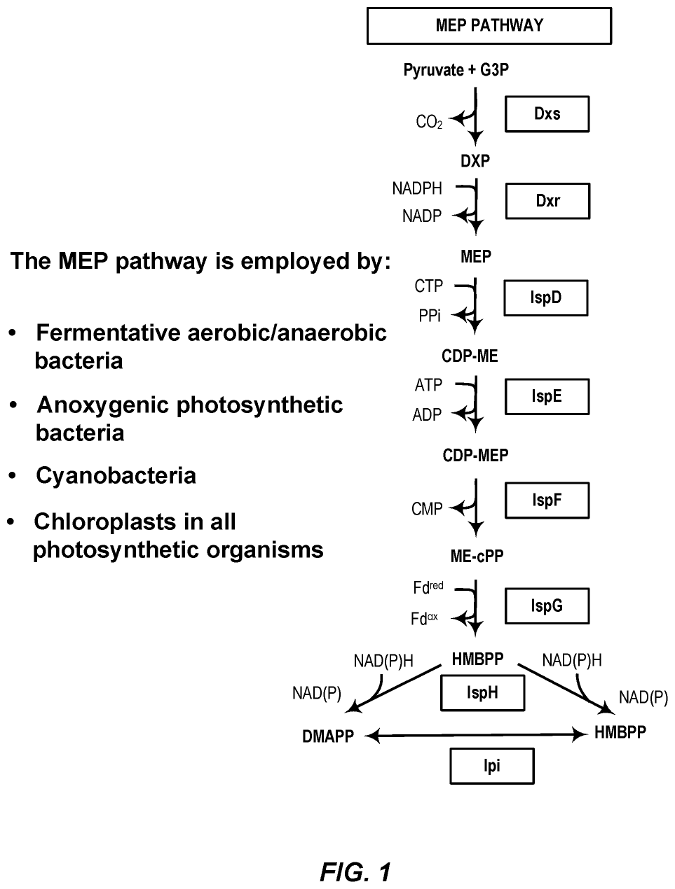

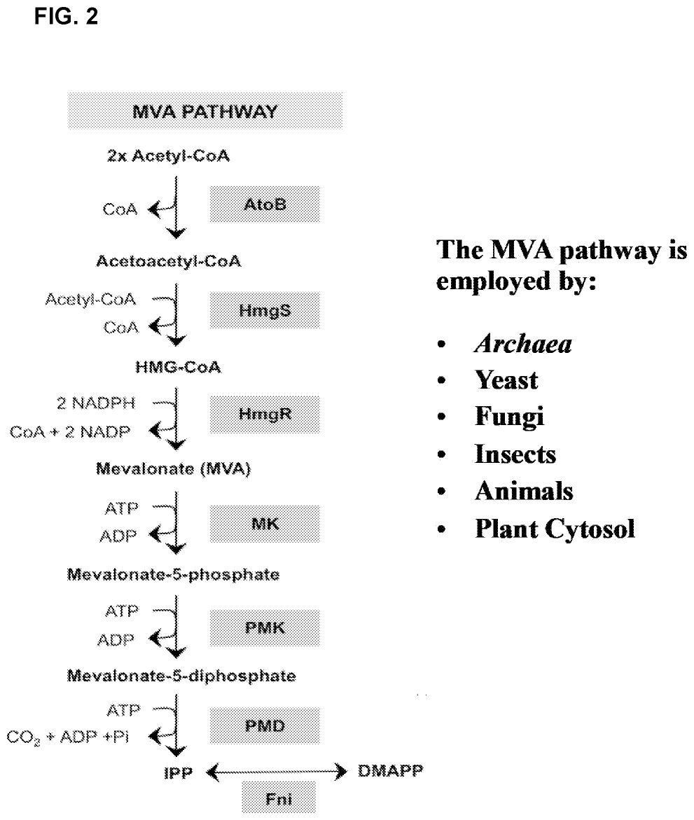

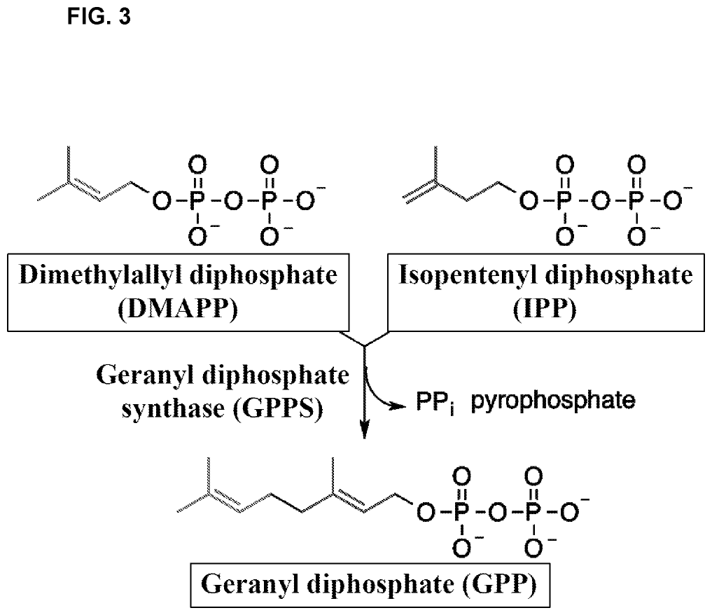

### DETAILED DESCRIPTION OF THE INVENTION

### 1. Introduction

The present invention provides methods and compositions for producing highly pure, easily isolatable cannabinoids in photosynthetic microorganisms that can be used for pharmaceutical, cosmetics-related, and other applications. The present methods provide numerous advantages for the production of cannabinoids, including that the cannabinoids can be produced constitutively from the natural photosynthesis of the cells, with no need to supplement growth media with antibiotics or organic nutrients, and that the produced cannabinoids can be readily harvested from the growth medium. Further, in some embodiments, the heterologous polynucleotides encoding the enzymes for the production of cannabinoids in the cells are integrated into the genome of the microorganisms, thereby avoiding potential difficulties resulting from the use of high-copy plasmids. Another advantage of the present methods is that cyanobacteria and other photosvnthetic microorganisms contain abundant thylakoid membranes of photosynthesis, which makes them particularly suitable for the expression and function of the transmembrane CBGAS enzyme.

The genetically modified photosynthetic microorganisms of the invention can be used commercially in an enclosed mass culture system to provide a source of cannabinoids which can be developed as biophamvaceutieals in the manifold therapeutic applications of cannabinoids currently employed or contemplated by the synthetic chemistry and pharmaceutical industries. For instance, the therapeutic potential of cannabidiol (CBD oil), a non-psychoactive substance, is currently being explored for a number of indications including for the treatment of pain, inflammatory diseases, epilepsy, anxiety disorders, substance abuse disorders, schizophrenia, cancer, and others.

### 2. Definitions

As used herein, the following terms have the meanings ascribed to them unless specified otherwise.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The terms “about” and “approximately” as used herein shall generally mean an acceptable degree of error for the quantity measured given the nature or precision of the measurements. Typically, exemplary degrees of error are within 20 percent (%), preferably within 10%, and more preferably within 5% of a given value or range of values. Any reference to “about X” specifically indicates at least the values X, 0.8X, 0.81X, 0.82X, 0.83X, 0.84X, 0.85X, 0.86X, 0.87X, 0.88X, 0.89X, 0.9X, 0.91 X, 0.92X, 0.93X, 0.94X, 0.95X, 0.96X, 0.97X, 0.98X, 0.99X, 1.01X, 1.02X, 1.03X, 1.04X, 1.05X, 1.06X, 1.07X, 1.08X, 1.09X, 1.1X, 1.11X, 1.12X, 1.13X, 1.14X, 1.15X, 1.16X, 1.17X, 1.18X, 1.19X, and 1.2X, Thus, “about X” is intended to teach and provide written description support for a claim limitation of. e.g., “0.98X.”

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and or deoxyinosioe residues (Batzer et al., Nucleic Acid Res. 19:5081 (1991); Ohtsuka et al., J. Biol. Chem. 260:2605-2608 (1985); and Rossolini et al, Mol. Cell Probes 8:91-98 (1994)).

The term “gene” refers to the segment of DNA involved in producing a polypeptide chain. It may include regions preceding and following the coding region (leader and trailer) as well as intervening sequences (introns) between individual coding segments (exons).

A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. The promoter can be a heterologous promoter, or an endogenous promoter, e.g., when a coding sequence is integrated into the genome and its expression is then driven by an adjacent promoter already present in the genome.

An “expression cassette” is a nucleic acid construct, generated recombinantly or synthetically, with a series of specified nucleic acid elements that permit transcription of a particular polynucleotide sequence in a host cell. An expression cassette may be pan of a plasmid, viral genome, or nucleic acid fragment. In some embodiments, an expression cassette includes a polynucleotide to be transcribed, operably linked to a promoter. The promoter can be a heterologous promoter. In the context of promoters operably linked to a polynucleotide, a “heterologous promoter” refers to a promoter dial would not be so operably linked to the same polynucleotide as found in a product of nature (e.g., in a wild-type organism). In some embodiments, the expression cassette comprises a coding sequence whose expression is designed to be driven by an endogenous promoter subsequent to integration into the genome.

As used herein, a first polynucleotide or polypeptide is “heterologous” to an organism or a second polynucleotide or polypeptide sequence if the first polynucleotide or polypeptide originates from a foreign species compared to the organism or second polynucleotide or polypeptide, or, if from the same species, is modified from its original form. For example, when a promoter is said to be operably linked to a heterologous coding sequence, it means that the coding sequence is derived from one species whereas the promoter sequence is derived from another, different species; or, if both are derived from the same species, the coding sequence is not naturally associated with the promoter (e.g., is a genetically engineered coding sequence).

“Polypeptide,” “peptide,” and “protein” are used interchangeably herein to refer to a polymer of amino acid residues. All three terms apply to amino acid polymers in which one or more amino acid residue is an artificial chemical mimetic of a corresponding naturally occurring amino acid, as well as to naturally occurring amino acid polymers and non-naturally occurring ammo acid polymers. As used herein, the terms encompass amino acid chains of any length, including full-length proteins, wherein the amino acid residues are linked by covalent peptide bonds.

“Conservatively modified variants” applies to both amino acid and nucleic acid sequences. With respect to particular nucleic acid sequences, “conservatively modified variants” refers to those nucleic acids that encode identical or essentially identical amino acid sequences, or where tlie nucleic acid dews not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

One of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles. In some cases, conservatively modified variants can have an increased stability, assembly, or activity.

The following eight groups each contain amino acids that are conservative substitutions for one another:

1) Alanine (A), Glycine (G);

2) Aspartic acid (D). Glutamic acid (E);

3) Asparagine (N), Glutamine (Q);

4) Arginine (R), Lysine (K);

5) Isoleucine (I), Leucine (L), Methionine (M), Valine (V);

6) Phenylalanine (F), Tyrosine (Y), Tryptophan (W);

7) Serine (S), Threonine (T); and

8) Cysteine (C), Methionine (M) (see, e.g., Creighton, Proteins, W. H. Freeman and Co., N. Y. (1984)).

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes. In the present application, amino acid residues are numbered according to their relative positions from the left most residue, which is numbered 1, in an unmodified wild-type polypeptide sequence.

As used in herein, tltc terms “identical” or percent “identity,” in the context of describing two or more polynucleotide or amino acid sequences, refer to two or more sequences or specified subsequences that are the same. Two sequences that are “substantially identical” have at least 60% identity, preferably 65%, 70%, 75%, 80%, 85%, 90%, 91%, 92%, 93, 94%, 95%, 96%, 97%, 98%, 99%, or 100% identity, when compared and aligned for maximum correspondence over a comparison window, or designated region as measured using a sequence comparison algorithm or by manual alignment and visual inspection where a specific region is not designated. With regard to polynucleotide sequences, this definition also refers to the complement of a test sequence. With regard to amino acid sequences, in some cases, the identity exists over a region that is at least about 50 amino acids in length, or more preferably over a region that is 75-100 amino acids in length. In some emodiments, percent identity is determined over the full-length of the amino acid or nucleic acid sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST 2.0 algorithm and the default parameters discussed below are used.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned.

An algorithm for determining percent sequence identity and sequence similarity is the BLAST 2.0 algorithm, which is described in Altschul et al., (1990) J. Mol. Biol. 215:403-410. Software for performing BLAST analyses is publicly available at the National Center for Biotechnology Information website, ncbi.nlm.nih.gov. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul el al., supra). These initial neighborhood word hits act as seeds for initiating searches to find longer HSPs containing them. The w ord hits are then extended in both directions along each sequence for as far as the cumulative alignment score can be increased. Cumulative scores are calculated using, for nucleotide sequences, the parameters M (reward score fora pair of matching residues: always >0) and N (penalty score for mismatching residues: always <0). For amino acid sequences, a scoring matrix is used to calculate the cumulative score. Extension of the word hits in each direction are halted when: the cumulative alignment score falls off by the quantity X from its maximum achieved value: the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences w ould occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

### 3. Photosynthetic Microorganisms

Any number of photosynthetic microorganisms can be used in the present methods. In particular embodiments, unicellular cyanobacteria are modified as described herein to produce cannabinoids. Illustrative cyanobacteria include, e.g., Synechocystis sp., such as strain Synechocystis PCO 6803; and Synechococcus sp., e.g., the thermophilic Synechococcus lividus; the mesophilic Synechococcus elongatus and Synechococcus 6301. and the euryhaline Synechococcus 7002. Multicellular, including filamentous cyanobacteria, may also be engineered to express the heterologous GPPS and cannabinoid biosynthesis operon genes in accordance with this invention, including, e.g., Gloeocapsa, as well as filamentous cyanobacteria such as Nostoc sp., e.g., Nostoc sp. PCC 7120, Nostoc sphaeroides); Anabaena sp., e.g., Anabaena variabilis; and Arthrospira sp. (“Spirulina”), such as Arthrospira platensis and Arthrospira maxima.

Algae, e.g., green microalgae, can also be modified to express GPPS and cannabinoid biosynthesis genes. Green microalgae are single cell oxygenic photosynthetic eukaryotic organisms that produce chlorophyll a and chlorophyll b. Thus, for example, in some embodiments, green microalgae such as Chlamydomonas reinhardtii, which is classified as Volvocales, Chlamydomonadaeeae, Scenedesmus obliquus, Nannochloropsis, Chlorella, Botryococcus braunii, Botryococcus sudeticus, Dunaliella salina, Haematococcus pluvialis, Chlorella fusca, and Chlorella vulgaris are modified as described herein to produce cannabinoids.

In some embodiments, photosynthetic microorganisms such as diatoms are modified. Examples of diatoms that can be modified to produce cannabinoids in accordance with this disclosure include Pheodactylum tricomutum; Cylindrotheca fusiformis; Cyclotella gamma; Nannochloropsis oceanica; and Thalassiosira pseudonana.

### 4. Polynucleotides

In the present disclosure, polynucleotides encoding a GPPS enzyme and encoding the enzymes of the cannabinoid biosynthesis pathway, e.g. AAE1, OLS, OAC, CBGAS, and one or more of CBDAS, THCAS, and CBCAS, are introduced into the photosynthetic microorganism, e.g., cyanobacteria.

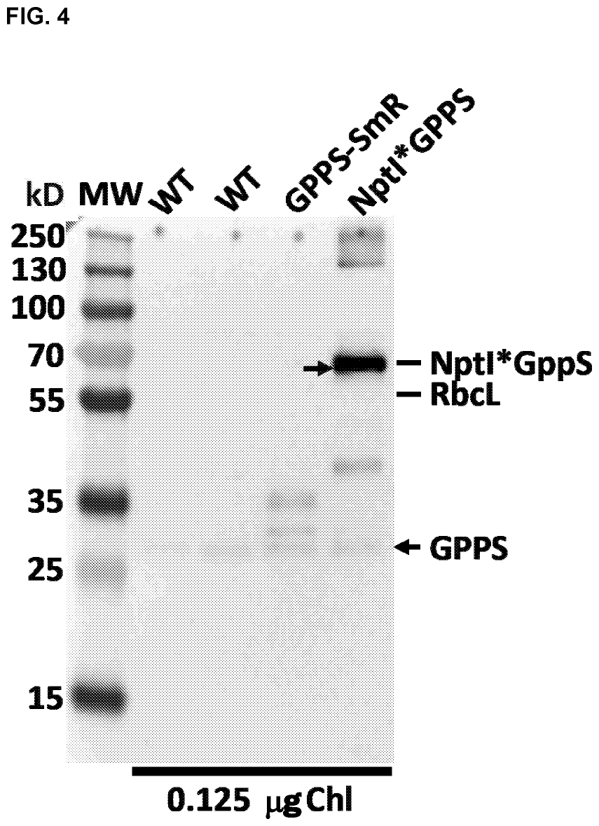

It is desirable that GPPS in particular is overexpressed to ensure a high level of GPP production in the cells. To obtain high levels of expression of GPPS or any of the present cannabinoid biosynthesis enzymes, one or more of the proteins may be expressed as a fusion construct. In preferred embodiments, the GPPS enzyme is expressed as a fusion construct, e.g., by fusing the polynucleotide encoding the GPPS polypeptide with the 3′ end of a leader nucleic acid sequence encoding a protein that is expressed in cyanobacteria at a level of at least 1% of the total cellular protein. For example, SEQ ID NO:1 discloses the DNA sequence of the nptI*GPPS fusion construct, comprising the GPPS gene from Picea abies (Noway spruce) fused to the nptI gene encoding the kanamycin resistance protein, codon optimized for high-level NptI*GPP protein expression and GPP pool size increase in the cyanobacterium Synechocystis (Betterle and Melis 2018). SEQ ID NO:2 discloses the amino acid sequence of this NptI*GPP fusion construct, the expression levels of which approach those of the abundant RbcL the large subunit of Rubisco in the modified cyanobacteria (FIG. 4).

The use of NptI and other fusion proteins to obtain high transgene yields in cyanobacteria and other photosynthetic microorganisms is described, e.g., in US Patent Application No. 2018/0171342 and in Application PCT/US2017034754, the entire disclosures of both of which ate incorporated herein by reference.

Other polynucleotides that may be employed in fusion constructs include, e.g., chloramphenicol acetyltrausferase polynucleotides, which confer chloramphenicol resistance, or polynucleotides encoding a protein that confers streptomycin, ampicilJin, or tetracycline resistance, or resistance to another antibiotic. In some embodiments, the leader sequence encodes less than the full-length of the protein, but typically comprises a region tliat encodes at least 25%, typically at least 50%, or at least 75%, or at least 90%, or at least 95%, or greater, of the length of the protein. In some embodiments, a polynucleotide variant of a naturally occurring antibiotic resistance gene is employed- As noted above, a variant polynucleotide need not encode a protein that retains the native biological function. A variant polynucleotide typically encodes a protein that has at least 80% identity, or at least 85% or greater, identity to the protein encoded by the wild-type gene, e.g., antibiotic resistance gene. In some embodiments, the polynucleotide encodes a protein that has 90% identity, or at least 95% identity, or greater, to the wild-type antibiotic resistance protein. Such variant polynucleotides employed as leader sequences can also be codon-optimizcd for expression in cyanobacteria. The percent identity is typically determined with reference to the length of the polynucleotide that is employed in the construct, i.e., the percent identity may be over the full length of a polynucleotide that encodes the leader polypeptide sequence, or may be over a smaller length, e.g., in embodiments where the polynucleotide encodes at least 25%, typically at least 50%, or at least 75%, or at least 90%, or at least 95%, or greater, of the length of the protein. A protein encoded by a variant polynucleotide sequence need not retain a biological function, although codons that are present in a variant polynucleotide are typically selected such that the protein structure relative to the wild-type protein structure is not substantially altered by the changed codon, e.g., a codon that encodes an amino acid that has the same charge, polarity, and or is similar in size to the native amino acid.

In some embodiments, the leader sequence encodes a naturally occurring cyanobacteria or other microorganismal protein that is expressed at a high level (e.g., more than 1% of the total cellular protein) in native cyanobacteria or the other microorganism of interest, i.e., the protein is endogenous to cyanobacteria or another microorganism of interest. Examples of such proteins include cpcB, cpcA, cpeA, cpeB, apcA, apcB, rbcL, rbcS, psbA, rpl, and rps. In some embodiments, the leader sequence encodes less than tltc full-length of the protein, but it typically comprises a region that encodes at least 25%, typically at least 50%, or at least 75%, or at least 90%, or at least 95%, or greater, of the length of the protein. Use of an endogenous microorganismaL e.g., cyanobacterial, polynucleotide sequence for constructing an expression construct in accordance with the invention provides a sequence that need not be codon-optimizcd, as the sequence is already expressed at high levels in the microorganism, e.g., cyanobacteria, although codon optimization is nevertheless possible. Examples of cyanobacterial or other microorganismal polynucleotides that encode cpcB, cpcA, cpeA, cpeB, ape A, apcB, rbcL, rbcS, psbA, rpl, or rps are available, e.g., at the www website genome.microbedb.jp/cyanobase.

The polynucleotide sequence that encodes the leader protein need not be 100% identical to a native cyanobacteria or other microorganismal polynucleotide sequence. A polynucleotide variant having at least 50% identity or at least 60% identity, or greater, to a native microorganismal, e.g., cyanobacterial, polynucleotide sequence, e.g., a native cpcB, cpcA, cpeA, cpeB. rbcL, rbcS, psbA, rpl, or ips polynucleotide sequence, may also be used, so long as the codons that vary relative to the native polynucleotide are codon optimized for expression in cyanobacteria or the microorganism being used and do not substantially disrupt the structure of the protein. In some embodiments, a polynucleotide variant that has at least 70% identity, at least 75% identity, at least 80% identity, or at least 85% identity, or greater to a native microorganismal, e.g., cyanobacterial polynucleotide sequence, e.g., a native cpcB, cpcA, cpeA, cpeB, rbcL, rbcS, psbA, rpl, or rps polynucleotide sequence, is used, again maintaining codon optimization for cyanobacteria or the microorganism of interest. In some embodiments, a polynucleotide variant that has least 90% identity, or at least 95% identity, or greater, to a native microorganismal, e.g., cyanobacterial, polynucleotide sequence, e.g., a native cpcB, cpcA, cpeA, cpeB, rbcL, rbcS, psbA, rpl, or rps polynucleotide sequence, is used. The percent identity is typically determined with reference the length of the polynucleotide that is employed in the construct, i.e., the percent identity may be over the full length of a polynucleotide that encodes the leader polypeptide sequence, or may be over a smaller length, e.g., in embodiments where the polynucleotide encodes at least 25%, typically at least 50%, or at least 75%, or at least 90%, or at least 95%, or greater, of the length of the protein. Although the protein encoded by a variant polynucleotide sequence as described herein need not retain a biological function, a codon that varies from the wild-type polynucleotide is typically selected such that the protein structure of the native cyanobacterial or other microorganisms I sequence is not substantially altered by the changed codon, e.g., a codon that encodes an amino acid that has the same charge, polarity, and or is similar in size to the native amino acid is selected.

In some embodiments, a protein that is expressed at high levels in the photosynthetic microorganism, e.g., cyanobacteria, is not native to the organism in which the fusion construct in accordance with the invention is expressed. For example, polynucleotides from bacteria or other organisms that are expressed at high levels in cyanobacteria or other photosynthetic microorganisms may be used as leader sequences. In such embodiments, the polynucleotides from other organisms are codon optimized for expression in the photosynthetic microorganism, e.g., cyanobacteria. In some embodiments, codon optimization is performed such that codons used with an average frequency of less than 12% by, e.g., Synechocystis are replaced by more frequently used codons. Rare codons can be defined, e.g., by using a codon usage table derived from the sequenced genome of the host cyanobacterial cell. Sec, e.g., the codon usage table obtained from Kazusa DMA Research Institute, Japan (website www.kazusa.or.jp codon) used in conjunction with software, e.g., “Gene Designer 2.0” software, from DNA 2.0 (website www.dna20.com ) at a cut-off thread of 15%.

In the context of the present invention, a protein, e.g., GPPS. that is “expressed at high levels” in photosynthetic microorganisms, e.g., cyanobacteria, refers to a protein that accumulates to at least 1% of total cellular protein as described herein. Such proteins, when fused at the N-terminus of a protein of interest to be expressed in cyanobacteria or other microorganisms, are also referred to herein as “leader proteins”, “leader peptides”, or “leader sequences”. A nucleic acid encoding a leader protein is typically referred to herein as a “leader polynucleotide” or “leader nucleic acid sequence” or “leader nucleotide sequence”.

In all cases, suitable leader proteins can be identified by evaluating the level of expression of a candidate leader protein in the photosynthctic microorganism of interest, e.g., cyanobacteria. For example, a leader polypeptide that does not occur in the wild type microorganism, e.g., cyanobacteria, may be identified by measuring the level of protein expressed from a polynucleotide codon optimized for expression in the microorganism, e.g., cyanobacteria, that encodes the candidate leader polypeptide. A protein may be selected for use as a leader polypeptide if the protein accumulates to a level of at least 1%. typically at least 2%, at least 3%, at least 4%, at least 5%, or at least 10%, or greater, of the total protein expressed in the cyanobacteria when the polynucleotide encoding the leader polypeptide is introduced into cyanobacteria and the cyanobacteria cultured under conditions in which the transgene is expressed. The level of protein expression is typically determined using SDS PAGE analysis. Following electrophoresis, the gel is scanned and the amount of protein determined by image analysis.

In one embodiment, a GPPS from Abies grandis is used, e g., as shown in SEQ ID NO:2, it will be appreciated, however, that any GPPS enzyme from any species that is capable of catalyzing the synthesis of GPP in the cells can be used, e.g., that is capable of catalyzing the production of GPP from 1PP and or DMAPP in the microorganisms.

In a particular embodiment, the photosvnthetic microorganisms are modified to overexprcss the GPP synthase (GPPS) gene, e.g., by use of a codon-optimized Abies grandis GPP synthase gene fused with the nptlkanamycin resistance DNA cassette (SEQ ID NO:1), in order to overexprcss the GPP synthase enzyme in the cell (SEQ ID NO:2). Such overexpression leads to greater amounts of the GPPS enzyme in the cell and enhancement of the GPP pool size in the microorganism, e.g., cyanobacteria. Polynucleotides that are functional variants, conservatively modified variants, and or that are substantially identical to SEQ ID NO:1), e.g., polynucleotides having 50%, 60%, 70%, 75%, 80%, 85%, 90%, 95%, 96%, 97%, 98%, 99%, or more identity to SEQ ID NO:1 one can be used, or a polynucleotide that encodes a protein having substantial identity, e.g., 50%. 60%, 70%, 75%, 80%, 85%, 90%, 95%, 96%, 97%, 98%, 99%, or more identity to SEQ ID NO:2, can be used, in particular when their presence in the cell leads to the generation of sufficient GPP for cannabinoid synthesis. In some embodiments, a polynucleotide having at least 95% identity to SEQ ID NO:l is used. In some embodiments, a polynucleotide that encodes a protein having at least 95% identity to SEQ ID NO:2 is used. In preferred embodiments, the GPPS are codon optimized for the cyanobacteria or other photosynthetic microorganism used in the method.

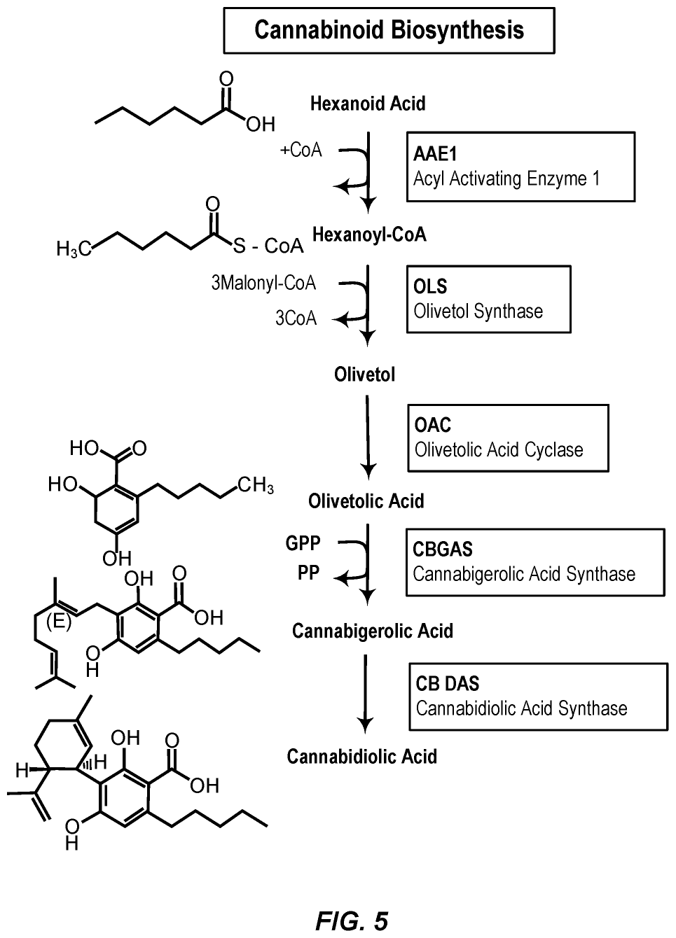

Genes encoding enzymes of the cannabinoid biosynthetic pathway are known and any such enzymes can be employed in the present methods, from any species, so long as they can be functionally expressed in the photosynthetic microorganisms, e.g., cyanobacteria, to effect the biosynthesis of the cannabinoids in the cells. A list of the genes needed to drive the eannabinoid biosynthetic pathway is shown in FIG. 5, and the associated alternative oxidocyclase enzymes (THCAS and CBCAS) that catalyze the oxidative cyclization of the monoterpene moiety of CBGA for the biosynthesis of Δ9-tetrahydrocannabinolic acid (Δ9-THCA) and catinabichromenic acid (CBGA), respectively, are provided in Table 1 (Carvalho et al. 2017). In general, in addition to the GPPS-encoding gene, genes are included for AAE1, OLS, OAC, and CBGAS, as well as for CBDAS, THCAS, or CBCAS, depending on whether CBDA, Δ9-THCA, or CBCA, respectively, is desired. It will be appreciated, however, that other combinations of genes are possible as well, for example GPPS, AAE1, OLS, OAC, and CBGAS if CBGA is desired, or GPPS, AAE1, OLS, OAC, as well as CBGAS, THCAS, and CBCA, if a combination of CBDA, Δ9-THCA, and CBCA is desired. The coding sequences for the individual genes in the eannabinoid biosynthesis pathway are indicated in SEQ ID NO:3, i.e., nucleotides 636-2798 for AEE1, nucleotides 2819-3973 for OLS, nucleotides 3994-4299 for OAC, nucleotides 4320-5507 for CBGAS, and nucleotides 5528-7162 for CBDAS. These sequences, or variants thereof as described herein, can be used individually or in any combination, e.g., within the same operon, to bring about eannabinoid synthesis in the photosynthetic microorganisms, e.g., cyanobacteria.

In one embodiment, a codon-optimized polynucleotide sequence in operon configuration of the cannabinoid biosynthesis pathway is used, leading to the synthesis of cannabidiolic acid. Such a polynucleotide is shown as SEQ ID NO:3, and includes coding sequences for AAE1, OLS, OAC, CBGAS, and CBDAS, whose polypeptide sequences are shown as SEQ ID NO:4, SEQ ID NO:5. SEQ ID NO:6, SEQ ID NO:7, and SEQ ID NO:8, respectively. Polynucleotides that are substantially identical to SEQ ID NO:3, e.g., that have at least 50%, 60%, 70%, 75%, 80% 85%, 90%, 95%, 96%, 97%, 98%, 99%, or more identity to SEQ ID NO:3, or that encode polypeptides that arc functional variants, e.g., conservatively modified variants, are substantially identical to any of SEQ ID NOS. 4, 5, 6, 7, or 8. can be used, e.g., that have at least 60%, 70%, 75%, 80% 85% 90%, 95% 96%, 97%, 98%, 99%, or more identity to SEQ ID Nos. 4, 5, 6, 7, or 8, can be used. In some embodiments, a polynucleotide that has at least 95% identity to SEQ ID NO:3 is used In some embodiments, a polynucleotide that encodes a protein having at least 95% identity to SEQ ID NO:4, 5, 6, 7, or 8 is used.

In embodiments where Δ9-THCA synthesis is desired, a polynucleotide comprising the sequence shown as SEQ ID NO:9 can be used, or a polynucleotide that is substantially identical to SEQ ID NO:9, e.g., at least 50%, 60%, 70%, 75%, 80%, 85%, 90%, 95%, 96%, 97%, 98%, 99%, or more identical to SEQ ID NO:9, or that encodes a polypeptide comprising the amino acid sequence shown as SEQ ID NO:10 can be used, or that encodes a functional variant polypeptide that is substantially identical to SEQ ID NO:10, e.g., at least 60%, 70%, 75%, 80%, 85%, 90%, 95%, 96%, 97%, 98%, 99% or more identical to SEQ ID NO:10. In some embodiments, a polynucleotide that has at least 95% identity to SEQ ID NO:9 is used. In some embodiments, a polynucleotide that encodes a protein having at least 95% identity to SEQ ID NO:10 is used. In a particular embodiment, when Δ9-THCA synthesis is desired, all of the biosynthesis genes are present within a single operon, e.g., as shown in SEQ ID NO:13, or using a polynucleotide having at least 50%, 60%, 70%, 75%, 80%, 85%, 90%, 95%, 96%, 97%, 98%, 99% or more identity to SEQ ID NO:13. In some embodiments, a polynucleotide having at least 95% identity to SEQ ID NO:13 is used.

In embodiments where CBCA synthesis is desired, a polynucleotide comprising the sequence shown as SEQ ID NO:11 can be used, or a polynucleotide that is substantially identical to SEQ ID NO:11, e.g., at least 50%, 60%, 70%, 75%, 80%, 85%, 90%, 95%, 96%, 97%, 98%, 99%, or more identical to SEQ ID NO:11, or that encodes a polypeptide comprising the amino acid sequence shown as SEQ ID NO:12. or that encodes a functional variant polypeptide that is substantially identical to SEQ ID NO:12, e.g., at least 60%, 70%, 75%, 80%, 85%, 90%, 95%, 96%, 97%, 98%, 99% or more identical to SEQ ID NO:12. In some embodiments, a polynucleotide having at least 95% identity to SEQ ID NO:11 is used. In some embodiments, a polynucleotide that encodes a protein having at least 95% identity to SEQ ID NO:12 is used. In a particular embodiment, when CBCA synthesis is desired, all of the biosynthesis genes are present within a single operon, e.g., as shown in SEQ ID NO:14, or using a polynucleotide having at least 50%, 60%, 70%, 75%, 80%, 85%, 90%, 95%, 96%, 97%, 98%, 99%, or more identity to SEQ ID NO:14. In some embodiments, a polynucleotide having at least 95% identity to SEQ ID NO:14 is used.

The genes encoding the enzymes within the biosynthesis pathway, i.e., AAE1, OLS, OAC, and CBGAS, as well as CBDAS, THCAS, and/or CBCAS, can be together present within a single operon (e.g., as in SEQ ID NO:3 in the case of CBDAS synthesis, in SEQ ID NO:13 in the case of Δ9-THCA synthesis, or in SEQ ID NO:14 in the case of CBCA synthesis) or present separately, or in any combination of individual genes and genes in an operon (e.g., AAE1, OLS, OAC, and CBGAS within an operon, and CBDAS separately). The gene encoding GPPS can also be included in the operon. The operon can include any combination of 2, 3, 4, 5, 6, 7 or 8 genes selected from GPPS, AAE1, OLS, OAC, CBGAS, CBDAS, THCAS, and CBCAS, and arranged in any order.

In some embodiments, one or more of the genes within the eannabinoid biosynthesis pathway, and or the GPPS gene, individually or as present within one or more operons, can be integrated into the genome of the host cell, e.g., via homologous recombination. In one embodiment, all of the transgencs used in the invention, i.e., GPPS, AAE1, OLS, OAC, CBGAS, and either CBDAS, THCAS, or CBCAS, are integrated into the host cell genome. In certain embodiments, however, one or more of the genes are present on an autonomously replicating vector.

| Enzyme Name | Abbreviation | Accession # | EC # | Reference |
|---|---|---|---|---|
| Acyl activating enzyme | AAE1 | AFD33345.1 | 6.2.1.1 | Sout et al. |
| 1 | | | | 2012 |
| Olivetol synthase | OLS | AB164375 | 2.3.1.206 | Taura et al. |
| | | | | 2012 |
| Olivetolic acid cyclase | OAC | AFN42527.1 | 4.4.1.26 | Gagne et al. |
| | | | | 2012 |
| Cannabigerolic acid | CBGAS | US8884100B2 | 2.5.1.102 | Fellermeier and |
| synthase | | | | Zenk 1998; |
| | | | | Page and |
| | | | | Boubakir 2012 |
| Cannabidiolic acid | CBDAS | AB292682 | 1.21.3.8 | Taura et al. |
| synthase | | | | 2007b |
| Tetrahydrocannabinolic | THCAS | AB057805 | 1.21.3.7 | Sirikantaramas |
| acid synthase | | | | et al. 2004 |
| Cannabichromenic acid | CBCAS | WO | 1.3.3 | Morimoto et al. |
| synthase | | 2015/196275 | | 1998; |
| | | A1 | | Page and Stout |
| | | | | 2015 |

In some embodiments, a ggaattaggaggttaattaa ribosome binding site (RBS) is positioned in front of the ATG start codon of one or more of the GPPS and/or cannabinoid biosynthesis pathway genes, in the photosynthctic microorganisms. This is designed to enhance the level of translation of all the genes encoded by the operon or construct. In some embodiments, the nucleic acids of the ggaattaggaggrtaattaa RBS are a codon-modified variant having at least 80% identity, typically at least 85% identity or 90%, 95%, 96%, 97%, 98%, 99%, or 100% identity to the ggaattaggaggttaattaa RBS nucleotides. In some embodiments, the nucleic acids have at least 95% identity to the ggaattaggaggttaattaa RBS nucleotides.

For the optimal expression of the GPPS and/or cannabinoid biosynthetic proteins in cyanobacteria or other photosynthetic microorganisms, the coding sequences can be codon optimized for expression in the cyanobacteria or other microorganisms. In some embodiments, codon optimization is performed such that codons used with an average frequency of less than, e.g., 12% in a species such as Synechocystis (or whichever species is being used to perform the methods) arc replaced by more frequently used codons. Rare codons can be defined, e.g., by using a codon usage table derived from the sequenced genome of the host cyanobacterial cell or other microorganism. See, e.g., the codon usage table obtained from Kazusa DNA Research Institute, Japan (website www.kazusa.or.jp/codon/) used in conjunction with software, e.g., “Gene Designer 2.0” software, from DNA 2.0 (website www.dna20.com/) at a cut-off thread of 15%.

The polynucleotides encoding the GPPS enzyme and or the cannabinoid biosynthesis operon are operably linked to one or more promoters capable of bringing about the expression of the GPPS and or cannabinoid biosynthesis enzymes in the cell at levels sufficient for the biosynthesis of cannabinoids. In some embodiments, the heterologous polynucleotide encoding the GPPS and/or the cannabinoid biosynthesis operon is operably linked to an endogenous promoter, e.g., the psbA2 promoter, e.g., by replacing the endogenous gene, e.g., the Synechocvstis psbA2 gene, with the codon-optimized GPPS-encoding gene or the cannabinoid biosynthesis operon via double homologous recombination.

In other embodiments, the GPPS-encoding polynucleotide and dr the cannabinoid biosynthesis operon are integrated into the genome and clones identified in which GPPS and or the enzymes of the cannabinoid biosynthesis pathway are produced at sufficiently high levels to obtain cannabinoid biosynthesis in the cell, and the polynucleotides encoding the promoter or promoters responsible for the expression identified by analyzing the 5′ sequences of the genomic clone or clones corresponding to the GPPS gene or the operon. Nucleotide sequences characteristic of promoters can also be used to identify the promoter.

In other embodiments, the GPPS-encoding polynucleotide and or the cannabinoid biosynthesis operon are operably linked to a heterologous promoter capable of driving expression in the cell. e.g., they are linked to a promoter within a vector before being introduced into the cell, and are then integrated together into the genome of the cell or are maintained together on an autonomously replicating vector. The promoters used can be either constitutive or inducible. In some embodiments, a promoter used for driving the expression of the GPPS or operon is a constitutive promoter. Examples of constitutive strong promoters for use in cyanobacteria or other photosynthesis microorganisms include, for example, the pshD1 gene or the basal promoter of the psbD2 gene, or the rbcLS promoter, which is constitutive under standard growth conditions. Other promoters that are active in cyanobacteria and other photosynthetic microorganisms are also known. These include the strong cpc operon promoter, the cpe operon and ape operon promoters, which control expression of phycobilisome constituents. The light-inducible promoters of the psbA1, psbA2, and psbA3 genes in cyanobacteria may also be used, as noted below. Other promoters that are operative in plants, e.g., promoters derived from plant viruses, such as the CaMV35S promoters, or bacterial viruses, such as the T7, or bacterial promoters, such as the PTrc, can also be employed in cyanobacteria or other photosynthetic microorganisms. For a description of strong and regulated promoters, any of which can be used in the present methods, e.g., promoters active in the cyanobacterium Anabaena sp. strain PCC 7120 and Synechocystis 6803, see e.g., Elhai, FEMS Microbiol Lett 114:179-184, (1993) and Formighieri, Planta 240:309-324 (2014). the entire disclosures of which are incorporated herein by reference.

In some embodiments, a promoter is used to direct expression of tltc inserted nucleic acids under the influence of changing environmental conditions. Examples of environmental conditions that may affect transcription by inducible promoters include anaerobic conditions, elevated temperature, or the presence of light. Promoters that are inducible upon exposure to chemical reagents are also used to express the inserted nucleic acids. Other useful inducible regulatory elements include copper-inducible regulatory elements (Mett et al., Proc. Natl. Acad. Sci USA 90:4567-4571 (1993); Furst et al., Cell 55:705-717 (1988)); copper-repressed petJ promoter in Synechocystis (Kuchmina et al 2012, J Biotechn 162:75-80): riboswitches, e.g. theophylline-dependent (Nakahira et al. 2013, Plant Cell Physiol 54:1724-1735; tetracycline and chlor-tetracycline-induciblc regulatory elements (Gatz et al., Plant J. 2:397-404 (1992); Röder et al., Mol Gen. Genet. 243:32-38 (1994): Gatz, Meth. Cell Biol 50:411-424 (1995)); ecdysone inducible regulatory elements (Christopherson et al., Proc. Natl. Acad. Sci. USA 89:6314-6318 (1992); Kreutzweiser et al, Ecotoxicol. Environ. Safety 28:14-24 (1994)): heat shock inducible promoters, such as those of the hsp70 dnaK genes (Takahashi et al., Plant Physiol 99:383-3% (1992); Yabe et al., Plant Cell Physiol. 35:1207-1219 (1994); Ueda et al., Mol Gen. Genet. 250:533-539 (1996)); and lac operon elements, which are used in combination with a constitutively expressed lac repressor to confer, for example. IPTG-inducible expression (Wilde et al., EMBO J. 11:1251-1259 (1992)). An inducible regulatory element also can be, for example, a nitrate-inducible promoter, e.g., derived from the spinach nitrite reductase gene (Back el al., Plant Mol. Biol. 17:9 (1991)), or a lighl-induciblc promoter, such as that associated with the small subunit of RuBP carboxylase or the LIICP gene families (Feinbaum et al, Mol. Gen. Genet. 226:449 (1991); Lam and Chua, Science 248:471 (1990)).

In some embodiments, the promoter is from a gene associated with photosynthesis in the species to be transformed or another species. For example such a promoter from one species may be used to direct expression of a protein in trams formed cyanobacteria or other photosynthetic microorganisms. Suitable promoters may be isolated from or synthesized based on known sequences from other photosynthetic organisms.

In certain embodiments, the methods comprise introducing expression cassettes that comprise nucleic acid single genes or operons encoding the genes of the cannabinoid biosynthetic pathway (FIG. 5) into the phoiosynthetie microorganism, e.g., cyanobacteria, wherein the operon is linked to a cpc promoter, or other suitable promoter; and culturing the microorganism, e.g., cyanobacteria under conditions in which the single gene or nucleic acids encoding the cannabinoid biosynthesis operon are expressed. In some embodiments, expression cassettes are introduced into the psbA2 gene locus, encoding the D1/32 kD reaction center protein of photosysiem-II, in which case the pshA2 promoter is the native cyanobacteria promoter. In other embodiments, expression cassettes are introduced into the glgA1 gene locus, encoding the glycogen synthase 1 enzyme, in which case the glgA1 promoter is the native cyanobacteria promoter.

In a particular embodiment, the polynucleotides encoding the GPPS enzyme, e.g., a GPPS fusion protein, and encoding the members of the cannabinoid biosynthesis pathway are introduced into the cells using a vector. Vectors comprising nptI*GPPS or the cannabinoid biosynthesis pathway operon nucleic acid sequences typically comprise a marker gene that confers a selectable phenotype on cyanobacteria or other microorganisms transformed with the vector. Such markers are known, for example markers encoding antibiotic resistance, such as resistance to chloramphenicol, kanamycin, spcctinomycin, erythromycin, G418, bleomycin, hygromycin, and the like.

Cell transformation methods and selectable markers for cyanobacteria and other photosynthetic microorganisms are well known in the art (Wirth, Mol. Gen. Genet., 216(1): 175-7, 1989; Koksharova, Appl. Microbiol. Biotechnol. 58(2): 123-37,2002: Thelwell et al., Proc. Natl. Acad. Sci. USA. 95:10728-10733, 1998: Formighieri and Melis, (Manta 248(4):933-946.2018: Betterle and Melis, ACS Synth Biol 7:912-921,2018). Transformation methods and selectable markers for are also well known (see, e.g., Sambrook et at., supra).

In some embodiments, an expression construct is generated to allow the heterologous expression of the nptI*GPPS and or the cannabinoid biosynthesis operon genes in Synechocystis through the replacement of the Synechocystis psbA2 gene with the codon-optimized nptI*GPPS or cannabinoid biosynthesis operon genes via double homologous recombination. In some embodiments, the expression construct comprises a codon-optimized nptI*GPPS or the cannabinoid biosynthesis operon genes gene operably linked to an endogenous cyanobacteria promoter. In some aspects, the promoter is the psbA2 promoter.

In some embodiments, the vector includes sequences for homologous recombination to insert the fusion construct at a desired site in a photosynthctic microorganismal, e.g., cyanobacterial, genome, e.g., such that expression of the polynucleotide encoding the fusion construct is driven by a promoter that is endogenous to the organism. Vectors to perform homologous recombination include sequences required for homologous recombination, such as flanking sequences that share homology with the target site for promoting homologous recombination.

In some embodiments, the photosynthctic microorganism, e.g., cyanobacteria, is transformed with an expression vector comprising the nptI*GPPS or the cannabinoid biosynthesis operon genes and an antibiotic resistance gene. Detailed descriptions are set forth, e.g., in Formighicri and Melis (Planta 240:309-324, 2014) Eglund et al (Sci Pep. 18;6:36640, 2016), and Wang et al. (ACS Synth. Biol. 7:276-286, 2018), which are incorporated herein by reference. Transformants are cultured in selective media containing an antibiotic to which an untransformed host cell is sensitive. Cyanobacteria, for example, normally have up to 100 copies of identical circular DNA chromosomes in each cell. The successful transformation with an expression vector comprising, e.g., the nptI*GPPS, the cannabinoid biosynthesis operon genes, and an antibiotic resistance gene normally occurs in only one or just a few, of the many cyanobacterial DNA copies. Hence, the presence of the antibiotic is necessary to encourage expression of the transgenic copy or copies of the DNA for cannabinoid production, in the absence of the selectable marker (antibiotic), the transgenic copy or copies of the DNA would be lost and replaced by wild-type copies of the DNA.

In some embodiments, cyanobacterial or other microorganismal transformants are cultured under continuous selective pressure conditions (presence of antibiotic over many generations) to achieve DNA homoplasmy in the transformed host organism. One of skill in the art understands that, to attain homoplasmy, the number of generations and length of time of culture varies depending on the particular culture conditions employed. Homoplasmy can be determined, e.g., by monitoring the genomic DNA composition in the cells to test for the presence or absence of wild-type copies of the cyanobacterial or other microorganismal DNA.

“Achieving homoplasmy” refers to a quantitative replacement of most, e.g., 70% or greater, or typically all, wild-type copies of the cyanobacterial DNA in the cell with the transformant DNA copy that carries the nptI*GPPS and the cannabinoid biosynthesis operon transgcnes. This is normally attained over time, under the continuous selective pressure (antibiotic) conditions applied, and entails the gradual replacement during growth of the wild-type copies of the DNA with the transgenic copies, until no wild-type copy of the cyanobacterial or other microorganismal DNA is left in any of the transformant cells. Achieving homoplasmy is typically verified by quantitative amplification methods such as genomic-DNA PCR using primers and/or probes specific for the wild-type copy of the cyanobacterial DNA. In some embodiments, the presence of wild-type cyanobacterial DNA can be detected by using primers specific for the wild-type cyanobacterial DNA and detecting the presence of, e.g., the native cpc operon, glgA1 or psbA2 genes. Transgenic DNA is typically stable under homoplasmy conditions and present in all copies of the cyanobacterial DNA.

In some embodiments, the photosynthetic microorganism, e.g., cyanobacteria, is cultured under conditions in which the light intensity is varied. Thus, for example, when a psbA2 promoter is used as a promoter to drive expression of nptI*GPPS or the cannabinoid biosynthesis operon genes, transformed cyanobacterial cultures can be grown at low light intensity conditions (e.g., 10-50 μmol photons m⁻²s⁻¹). then shifted to higher light intensity conditions (e.g., 500-1,000 μmol photons m⁻²s⁻¹). ThepsbA2 promoter responds to the shift in light intensity by up-regulating the expression of the nptI*GPPS fusion construct transgenc and the cannabinoid biosynthesis operon genes in Synechocystis, typically at least about 10-fold. In other embodiments, cyanobacterial cultures can be exposed to increasing light intensity conditions (e.g., from 50 μmol photons m⁻²s⁻¹ to 2,500 μmol photons m⁻²s⁻¹) corresponding to a diurnal increase in light intensity up to full sunlight. The psbA2 promoter responds to the gradual increase in light intensity by up-regulating the expression of the nptI*GPPS or the cannabinoid biosynthesis operon genes in Synechocystis in parallel with the increase in light intensity.

In some embodiments, cyanobaeterial or other microbial cultures arc cultured under conditions in which the cell density is high and transmitted light intensity through the culture is steeply attenuated. Thus, for example, when a cpc promoter is used as a promoter to drive expression of nptI*GPPS or the cannabinoid biosynthesis operon genes, transformed cyanobaeterial cultures can be grown at cell density conditions in which incident light intensity is high but irradiance entering the culture is quantitatively absorbed due to the high density of the culture, a desirable property for commercial exploitation (e.g. 1 g dry cell biomass per L culture). The cpc promoter responds to the diminishing light intensity within the culture by up-regulating the expression of the associated nptI*GPPS or the cannabinoid biosynthesis operon genes in Synechocystis, typically at least about 10-fold. Thus, the cpc promoter responds to the gradual decline in effective light intensity transmitted through ihe culture by up-regulating the expression of the nptI*GPPS or the cannabinoid biosynthesis operon genes in Synechocystis in a function antiparallei with the lowering in light intensity.

### 5. Production of Cannabinoids in Cyanobacteria or Other Photosynthetic Microorganisms

To produce cannabinoids, transformant photosynthetic microorganisms, e.g., cyanobacteria, are grown under conditions in which the heterologous nptI*GPPS and the cannabinoid biosynthesis operon genes are expressed. Methods of mass culturing photosynthetic microorganisms, e.g., cyanobacteria, are known to one skilled in the art. For example, cyanobacteria or other microorganisms can be grown to high cell density in photobioreactors (see. e.g., Lee et al., Biotech. Bioengineering 44:1161-1167, 1994; Chaumont, J Appl. Phycology 5:593-604, 1990). Examples of photobioreactors include cylindrical or tubular bioreactors, sec, e.g., U.S. Pat. Nos. 5,958,761, 6,083,740, US Patent Application Publication No. 2007 0048859; WO 2007/011343, and WO2007/098150. High density photobioreactors are described in, for example, Lee. et al., Biotech. Bioengineering 44: 1161-1167, 1994. Other photobioreaetors suitable for use in the invention are described, e.g., in WO2011 034567 and references cited therein, e.g., in the background section. Photobioreactor parameters that can be optimized, automated and regulated for production of photosynthctic organisms are further described in Puiz (Appl. Microbiol Biotechnol 57:287-293, 2001). Such parameters include, but are not limited to, materials of construction, efficient light delivery into the reactor lumen, light path, layer thickness, oxygen released, salinity and nutrients, pH, temperature, turbulence, optical density, and the like.

Transformant photosynthctic microorganisms, e.g., cyanobacteria, that express a heterologous nptI*GPPS and the cannabiuoid biosynthesis operon genes can be grown under mass culture conditions for the production of cannabinoids. In typical such embodiments, the transformed organisms are grown in biorcactors or fermenters that provide an enclosed environment. For example, in some embodiments for mass culture, the cyanobacteria are grown in enclosed reactors in quantities of at least about 100 liters, or 500 liters, often of at least about 1000 liters or greater, and in some embodiments in quantities of about 1,000,000 liters or more. Large-scale culture of transformed cyanobacteria that comprise a heterologous nptI*GPPS and the cannabinoid biosynthesis operon genes where expression is driven by a light sensitive promoter, such as a psbA2 or cpc promoter, is typically carried out in conditions where the culture is exposed to natural sunlight. Accordingly, in such embodiments, appropriate enclosed reactors are used that allow light to reach the cyanobacteria or other microbial culture.

Growth media for culturing the photosynthetic microorganism, e.g., cyanobacteria, transformants are well known in the art. For example, cyanobacteria or other microorganisms may be grown on solid BG-11 media (see, e.g., Rippka el at., J. Gen Microbiol. 111:1-61, 1979). Alternatively, they may be grown in liquid media (see. e.g., Bentley, F K and Melis, A. Biotechnol. Bioeng. 109:100-109, 2012). In typical embodiments for production of cannabinoids, liquid cultures are employed. For example, such a liquid culture may be maintained at. e.g., about 25° C. to 35° C. under a slow stream of constant aeration and illumination, e.g., at 20 μmol photons m⁻²s⁻¹ or greater. In certain embodiments, an antibiotic, e.g., chloramphenicol, is added to the liquid culture. For example, chloramphenicol may be used at a concentration of 15 μg/ml.

In some embodiments, photosynthetic microorganisms, e.g., cyanobacteria, transformants are grown photoautotrophically in a gaseous aqueous two-phase photobioreactor (see, e.g., U.S. Pat. No. 8,993,290; also Bentley, F K and Melis, A. Biotechnol Bioeng. 109:100-109 (2012). In some embodiments, the methods of the present invention comprise obtaining cannabinoids using a diffusion-based method for spontaneous gas exchange in a gaseous aqueous two-phase photobioreactor (see, e.g., U.S. Pat. No. 8,993,290). In particular aspects of the method, carbon dioxide is used as a feedstock for the photosynthctic generation of cannabinoids in cell culture, and the headspace of the biorcacior is filled with 100% CO₂ and sealed. This allows diffusion-based CO₂ uptake and assimilation by the cells via photosynthesis, and concomitant replacement of the CO₂ in the headspace with O₂. In some embodiments, the photosynthetically generated cannabinoids accumulate as a non-miscible product floating on the top of the liquid culture.

In particular embodiments, a gaseous aqueous two-phase photo-bioreactor is seeded with a culture of microbial, e.g., cyanobacterial, cells and grown under continuous illumination, e.g., at 75 μmol photons m⁻²s⁻¹, and continuous bubbling with air. Inorganic carbon is delivered to the culture in the form of aliquots of 100% CO₂ gas, which is slowly bubbled through the bottom of the liquid culture to fill the bioreactor headspace. Once atmospheric gases are replaced with 100% CO₂, the headspace of the reactor is scaled and the culture is incubated, e.g., at about 25° C. to 40° C. under continuous illumination, e.g., of 50 μmol photons m⁻²s⁻¹ or greater up to full sunlight. Slow continuous mechanical mixing is also employed to keep cells in suspension and to promote balanced cell illumination and nutrient mixing into the liquid culture in support of photosynthesis and biomass accumulation. Uptake and assimilation of headspace CO₂ by cells is concomitantly exchanged for O₂ during photoautotrophic growth. The sealed bioreactor headspace allows for the trapping, accumulation and concentration of photosymhetically produced cannabinoids.

In some embodiments, the photoautotrophic cell growth kinetics of the microbial, e.g., cyanobacteria, transformants are similar to those of wild type cells. In some embodiments, the rates of oxygen consumption during dark respiration are about the same in wild-type cyanobacteria or other photosynthetic microbial cells. In some embodiments, the rates of oxygen evolution and the initial slopes of photosynthesis as a function of light intensity are comparable in wild-type Synechocystis cells and Synechocystis transformants, when both are at sub-saturating light intensities between 0 and 250 μmol photons m⁻²s⁻¹.

Cannabinoids produced by the modified cyanobacteria or other microorganisms can be harvested using known techniques. Cannabinoids are not miscible in water and they rise to and float at the surface of the microorganism growth medium. Accordingly, in some embodiments, cannabinoids are siphoned off from the surface of the growth medium and sequestered in suitable containers, or floating cannabinoids are skimmed from the surface of the liquid phase of the culture and isolated in pure form. In some embodiments, the photosyntheticallv produced non-miscible cannabinoids in liquid form are extracted from the liquid phase by a method comprising overlaying a solvent such as heptane, decane, or dodecane on top of the liquid culture in the bioreactor, incubating at, e.g., room temperature for about 30 minutes or longer; and removing the solvent, e.g., heptane, layer containing the cannabinoids.

In some embodiments, the cannabinoids produced by the modified cyanobacteria or other microorganisms are extracted from the interior of the cells. For example, the cells can be isolated, e.g., by centrifugation at 5,000 g for 20 minutes, and then resuspended in, e.g., distilled water. The resuspended cells can then be disintegrated, e.g., by forcing the cells through a French press (e.g., at 1500 psi), by sonication, or treating them with glass beads. The resulting crude cell extract can then be centrifuged, e.g., at 14,000 g for 5 minutes, and the supernatant (or “disintegrated cell suspension”) used for extraction of the cannabinoids. In one embodiment, the cannabinoids are extracted by first mixing the disintegrated cell suspension with a strong acid and a salt, e.g., H₂SO₄ and NaCl, to ease the separation of the aqueous phase from the solvent phase, and to force hydrophobic molecules such as CBD to migrate to the solvent phase. Such methods are known in the art. In some embodiments, H₂O₄ and NaCl are added at a vohune-to-volume ratio of about [cell suspension/H₂SO₄/NaCl=3/0.12/0.5]. The suspension can then be extracted with one or more organic solvents, e.g., hexane, heptane, ethyl acetate, acetone, methanol, ethanol, and/or propanol. In some embodiments, the cannabinoids are obtained from the cultured modified cyanobacteria or other microorganisms by freeze drying the cells and subsequently extracting them with one or more organic solvents, e.g., methanol, acetonitrile, ethyl acetate, acetone, ethanol, propanol, hexane, and or heptane. In some embodiments, following extraction of the cannabinoids from the disintegrated or freeze-dried cells, the organic layer can tlien be separated from the aqueous medium and dried by solvent evaporation, leaving the cannabinoids in pure form. Jlte purified cannabinoids can then be resuspended and analyzed, e.g., using GC-MS. GC-FID, or absorbance spectrophotometry such as UV spectrophotometry.

### EXAMPLES

The examples described herein are provided by way of illustration only and not by way of limitation. One of skill in the art recognizes a variety of non-critical parameters that could be changed or modified to yield essentially similar results.

### Example 1

### Cannabinoid Production Using Genetically Engineered Cyanobacteria

The present invention provides methods and compositions for the genetic modification of cyanobacteria to confer upon these microorganisms the ability to produce cannabinoids upon heterologous expression of a nptI*GPPS fusion construct from Norway spruce (Picea abies) and the eannabinoid biosynthesis operon genes from cannabis (Cannabis saliva) or a variant thereof. In some embodiments, the invention provides for production of cannabinoids in gaseous-aqueous two-phase photobioreactors and results in the renewable generation of a hydrocarbon bio-product which can be used, e.g., for chemical synthesis, or for pharmaceutical, medical, and cosmetics-related applications. This example illustrates the expression of the heterologous nptI*GPPS and eannabinoid biosynthesis operon genes for the production of cannabinoids.

This example further illustrates that cannabinoids can be continuously (constitutively) generated in cyanobacteria transformants that express the heterologous nptI*GPPS fusion construct and eannabinoid biosynthesis operon genes. Further, this example demonstrates that cannabinoids can spontaneously diffuse out of cyanobacteria transformants and into the extracellular water phase, and be collected from the surface of the liquid culture as a water-floating product. This example also demonstrates that this strategy for production of cannabinoids alleviates product feedback inhibition, product toxicity to the cell, and the need for labor-intensive extraction protocols.

Photosynthetic microorganisms, with the cyanobacterium Synechocystis sp. PCC6803 as the model organism, were genetically engineered to express a nptI*GPPS fusion construct and eannabinoid biosynthesis operon genes, thereby endowing upon them the property of eannabinoid production (FIG. 5). Genetically modified strains were used in an enclosed mass culture system to provide renewable cannabinoids that are suitable as feedstock in chemical synthesis and the pharmaceutical, medical, and cosmetics-rclatcd industries. The cannabinoids were spontaneously emitted by the cells into the extracellular space, after which they floated to the surface of the liquid phase where they were easily collected without imposing any disruption to the growth;productivity of the cells. Hie genetically modified cyanobacteria remained in a continuous growth phase, constituti vely generating and emitting cannabinoids. The example further provides a codon-optimized nptI*GPPS fusion construct and eannabinoid biosynthesis operon genes for improved yield of cannabinoids in photosynthctic cyanobacteria, e.g., Synechocystis.

### Materials and Methods

### Strains and Growth Conditions

The E. coli strain DH5α was used for routine subcloning and plasmid propagation, and was grown in LB media with appropriate antibiotics as selectable markers at 37° C., according to standard protocols. The glucose tolerant cyanobacterial strain Synechocystis sp. PCC 6803 (Williams, JGK (1988) Methods Enzymol. 167:766-768) was used as the recipient strain in this study, and is referred to as the wild type. Wild type and transformant strains were maintained on solid BG-11 media supplemented with 10 mM TES-NaOH (pH 8.2), 0.3% sodium thiosulfate, and 5 mM glucose. Where appropriate, chloramphenicol kanamycin, spectinomycin, or erythromycin were used at a concentration of 15-30 μg/mL. Liquid cultures were grown in BG-11 containing 25 mM sodium phosphate buffer, pH 7.5. Liquid cultures for inoculum putposes and for pbotoautotrophic growth experiments and SDS-PAGE analyses were maintained at 25° C. under a slow stream of constant aeration and illumination at 20 μmol photons m⁻²s⁻¹. The growth conditions employed when measuring the production of cannabinoids from Synechocystis cultures are described below in the cannabinoid production assays section.

Codon-Use Optimization of the Heterologous nptI*GPPS Fusion Construct and Cannabinoid Biosynthesis Operon Genes for Expression in Synechocystis sp. PCC 6803 and Escherichia coli

The nucleotide and translated protein sequences of the heterologous nptI*GPPS fusion construct and cannabinoid biosynthesis operon genes were obtained from the NCBI GenBank database (National Center for Biotechnology Information: see, e.g., Table 1). The protein sequences of the heterologous nptI*GPPS fusion construct and cannabinoid biosynthesis operon gene products were obtained from the NCBI GenBank database (National Center for Biotechnology Information; see, e.g., SEQ ID NOS:2, 4-8. The codon-use of the resulting eDNAs was then optimized for expression in Synechocystis sp. PCC 6803 and E. coli (SEQ ID NO:1 and SEQ ID NO:3) To maximize the expression of the heterologous nptI*GPPS fusion construct and cannabinoid biosynthesis operon genes in Synechocystis sp. PCC 6803 and E. coli, these protein sequences were back-translated and codon-optimized according to the frequency of the codon usage in Synechocystis sp. PCC 6803. The codon-optimization process was performed based on the codon usage table obtained from Kazusa DNA Research institute, Japan (see, e.g., the www website kazusa.or.jp/codon/), and using the “Gene Designer 2.0” software from DNA 2.0 (see, e.g., the www website dna20.com). The codon-optimized genes were designed with appropriate restriction sites llanking the sequences to aid subsequent cloning steps.

Samples for SDS-PAGE analyses were prepared from Synechocystis cells resuspended in phosphate buffer pH 7.4 at a concentration of 0.12 mg/ml chlorophyll. Hie suspension was supplemental with 0.05% w/v lysozyme (Thermo Scientific) and incubated with shaking at 37° C. for 45 min. Cells were then pelleted at 4,000 g, washed twice with fresh phosphate buffer and disrupted with a French Pressure chamber (Aminco, USA) at 1500 psi in the presence of 1 mM PMSF. Soluble protein was separated from the total cell extract by centrifugation at 21,000 g and removed as the supernatant fraction. Samples for SDS-PAGE analysis were solubilized with 1 volume of 2× denaturing protein solubilization buffer (0.25 M Tris, pH 6.8,7% w/v SDS, 2 M urea, and 20% glycerol). In addition, all samples in denaturing solutions were supplemented w ith a 5% (v/v) of β-mercaptoethanol and centrifuged at 17,900 g for 5 min prior to gel loading. For Western blot analyses. Any kD™ (BIO-RAD) precast SDS-PAGE gels were utilized to resolve proteins, which were then transferred to PVDF membrane (Immobilon-FL 0.45 μm, Millipore, USA) for immunodetection using the rabbit immune serum containing specific polyclonal antibodies against the proteins of interest. Cross-reactions were visualized by the Supersignal West Pico Chemiluminiscent substrate detection system (Thermo Scientific, USA).

### Chlorophyll Determination, Photosynthetic Productivity and Biomass Quantitation

Chlorophyll a concentration in cultures was determined spectrophotometrically in 90% methanol extracts of the cells according to Meeks and Castenholz (Arch. Mikrobiol. 78:25-41,1971). Photosynthetic productivity of the cultures was tested polarographically with a Clark-type oxygen electrode (Rank Brothers, Cambridge, England). Cells were harvested at the mid-exponential growth phase, and maintained at 25° C. in BG11 containing 25 mM HEPES-NaOH, pH 7.5, at a chlorophyll a concentration of 10 μg/mL. Oxygen evolution was measured at 25° C. in the electrode upon yellow actinic illumination, which was defined by a CS 3-69 long wavelength pass cutoff filter (Corning, Corning, N.Y.). Photosynthetic activity of a 5 mL aliquot of culture was measured at varying actinic light intensities in the presence of 15 mM NaHCO₃ pH 7.4, added to provide inorganic carbon substrate and thereby facilitate generation of the light saturation curve of photosynthesis. Culture biomass accumulation was measured gravimetrically as dry cell weight, where 5 mL samples of culture were filtered through 0.22 μm Millipore filters, washed three times to remove nutrient salts. Subsequently, the immobilized cells were dried at 90° C. for 6 h prior to weighing the dry cell weight.

### Cannabinoid Production and Quantification Assays

Synechocystis cultures for cannabinoid production were grown photoautotrophicaliy in 1 L gaseous/aqueous two-phase photobioreactors, described in detail by Bentley and Melis (2012; Biotechnol Bioeng. 109:100-109). Bioreactors were seeded with a 700 ml culture of Synechocystis cells at an OD730 nm of 0.05 in BG11 medium containing 25 mM sodium phosphate buffer, pH 7.5, and grown under continuous illumination at 75 μmol photons m⁻²s⁻¹, and continuous bubbling with air until an OD730 nm of approximately 0.5 was reached, inorganic carbon was delivered to the culture in the form of 500 mL aliquots of 100% CO₂ gas. which was slowly bubbled though the bottom of the liquid culture to fill the bioreactor headspace. Once atmospheric gases were replaced with 100% CO₂, the headspace of the reactor was scaled and the culture was incubated under continuous illumination of 150 μmol photons m⁻²s⁻¹ at 35° C.. Slow continuous mechanical mixing was employed to keep cells in suspension and to promote balanced cell illumination and nutrient mixing into the liquid culture in support of photosynthesis and biomass accumulation. Uptake and assimilation of headspace CO₂ by cells was concomitantly exchanged for O₂ during photoautotrophic growth. The sealed biorcactor headspace allowed for the trapping, accumulation and concentration of photosyntheticallv produced cannabinoids, as liquid compounds Boating on the surface of the aqueous phase.

Photosynthetically produced non-miseibJe cannabinoids in liquid form were extracted from the liquid phase upon overlaying 20 mL heptane on top of the liquid culture in the bioreactor, and upon incubating for 30 min, or longer, at room temperature. The heptane layer was subsequently removed and analyzed by GC-FID, GC-MS, and absorbance spectrophotometry for the detection of cannabinoids by comparison with the liquid of a standard also dissolved in heptane. GC-FID analysis was performed with a Shimadzu 2014 instrument. GC-MS analyses were performed with an Agilent 6890GC 5973 MSD equipped with a DB-XLB column (0.25 mm i.d.×0.25 μm×30 m, J & W Scientific). Oven temperature was initially maintained at 40° C. for 4 min, followed by a temperature increase of 5° C./min to 80° C., and a carrier gas (helium) flow rate of 1.2 ml per minute. Absorbance spectrophotometry analysis was carried out with a Shimadzu UV-1800 spectrophotometer.

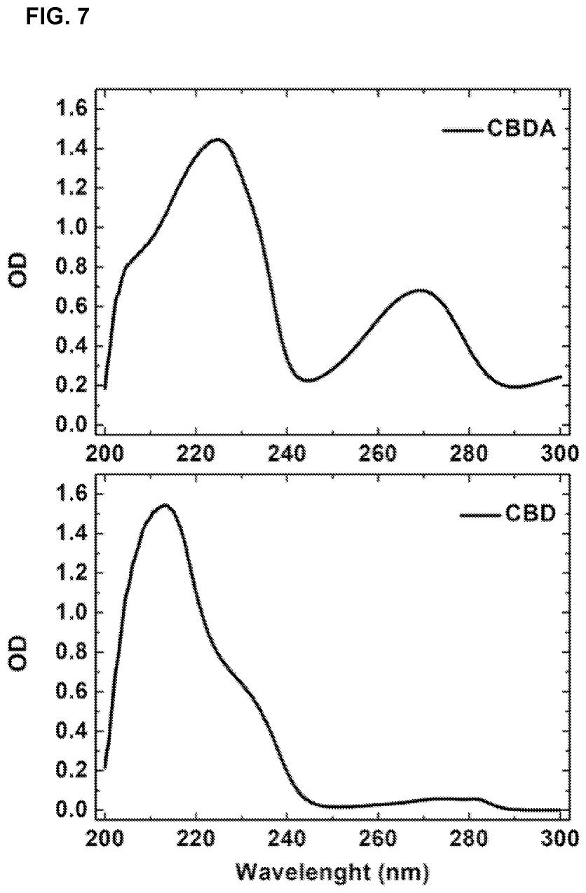

Accumulation of cannabinoids in the liquid phase was quantified spectrophotometricaily according to known absorbance spectra and extinction coefficients of cannabidiol and cannabidiolic acid in organic solvents (e.g., see FIG. 7). The majority of photosynthctically produced cannabinoids accumulated as a liquid floating over the aqueous phase of the biorcactor. A small amount of cannabinoids was initially retained within the cells, but was teased out of the cells by the 20 mL of heptane organic overlayer. Therefore, the non-miscible, heptane-extracted cannabinoids were used to generate the absorption spectra of cannabidiol and cannabidiolic acid in heptane for quantification purposes.

### Results

The native Escherichia coli K12 nptI gene, the Picea abies (Norway spruce) GGPS gene, and the native Cannabis saliva cannabinoid biosynthesis genes have codon usage different from that preferred by photosynthetic microorganisms, e.g., cyanobacteria and microalgae. The unicellular cyanobacteria Synechocystis sp. were used as a model organism in the development of the present invention. De novo codon-optimized nptI, GGPS, and Cannabis sativa cannabinoid biosynthesis genes were designed and synthesized. In the optimized version of these genes, SEQ ID NO:1 and SEQ ID NO:3, the codon usage was adapted to eliminate codons rarely used in Synechocystis, and to adjust the GC/AT ratio to that of the host. Rare codons were defined using a codon usage table derived from the sequenced genome of Synechocystis. The SEQ ID NO:1 and SEQ ID NO:3 sequences used in this example were: the codon-optimized nptI. GGPS, and Cannabis sativa cannabinoid biosynthesis genes for expression in Synechocystis.

SDS-PAGE analyses and immuno-detection of the nptI, GGPS, and Cannabis sativa cannabinoid biosynthesis gene products, using specific polyclonal antibodies raised against the E. coli-cxpressed recombinant protein, confirmed the presence of these recombinant proteins in Synechocystis (e.g., FIG. 4). These results clearly showed that the recombinant nptI, GGPS, and Cannabis sativa cannabinoid biosy nthesis gene products were expressed in Synechocystis transformants, and that they accumulated as internal proteins in the cell.

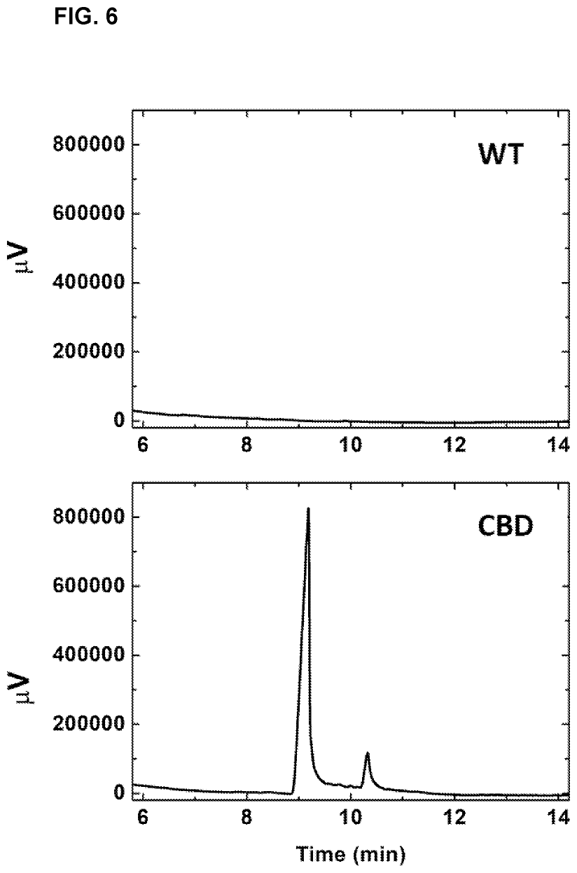

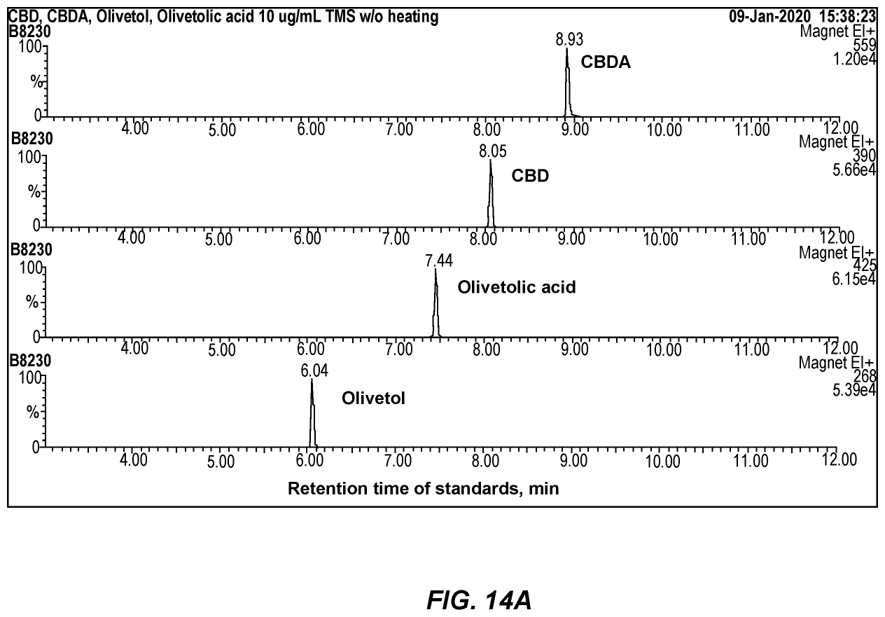

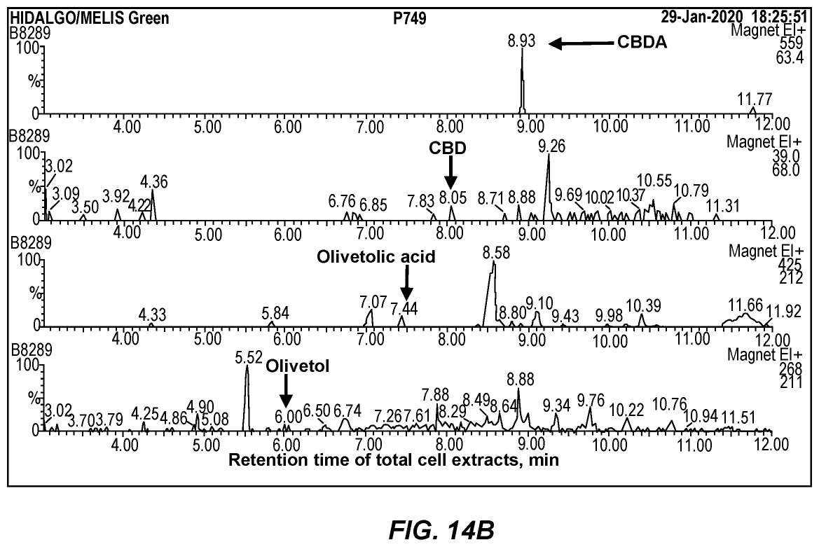

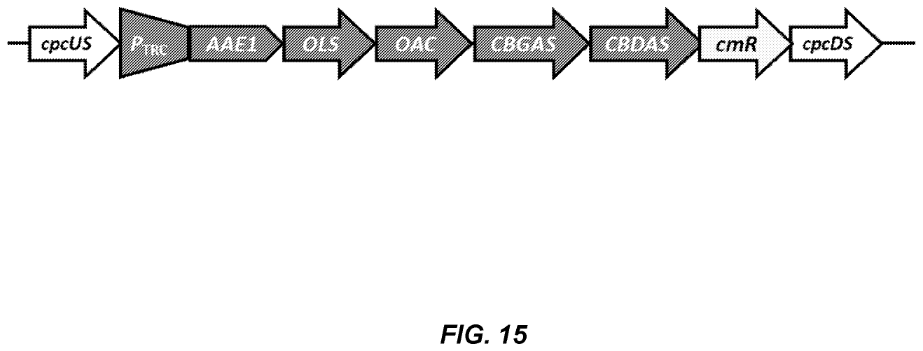

The above results demonstrated that Synechocystis can be used for heterologous transformation using the nptI, GGPS gene, and the Cannabis sativa cannabinoid biosynthesis genes, and that such transformants expressed and accumulated the respective proteins in their cytosol. To determine whether the expressed recombinant proteins are metabolically competent, wild type and transformants were cultivated under the conditions of the gaseous aqueous two-phase bioreactor (Bentley FK and Melis A, (2012). Biotechnol Bioeng. 109:100-109 ), with 100% CO₂ gas occupying the headspace prior to sealing the reactor to allow autotrophic biomass accumulation. Samples were obtained fr ont the surface of liquid cultures (to detect non-miscible liquid canoabinoids floating on top of the aqueous phase) and analyzed by GC-FID (e.g., FIG. 6) or GC-MS (e.g., FIGS. 14A-14B).

Quantification of cannabinoids in the heptane-extracted samples from the nptI*GPPS fusion construct and cannabinoid biosynthesis operon transformants was dctennined according to the Beer-Lambert Law, using the absorbance values measured at 250 nm and the known molar extinction coefficient of cannabinoids. During 48 h of active photoautotrophic growth in the presence of CO₂ in a sealed gaseous aqueous two-phase bioreactor, a 700 ml culture of nptI*GPPS fusion construct and cannabinoid biosynthesis operon transformants produced cannabinoids in the form of a non-miscibie product tloating on the surface of thre culture.

### Discussion

This example illustrates the production of cannabinoids in a system where the same organism serves both as photo-catalyst and producer of ready-made compounds. A number of guidelines have been applied in the endeavor of cyanobacterial cannabinoid biosynthesis, as they pertain to the selection of organisms and, independently, to the selection of potential product. Criteria for the selection of organisms include the solar-to-product energy conversion efficiency, which must be as high as possible. This important criterion is better satisfied with photosynthetic microorganisms than with crop plants (Melis A., Plant Science 177:272-280, 2009). Criteria for the selection of potential commodity products include (i) the commercial utility of the compound and (ii) the question of product separation from the biomass, which enters prominently in the economics of the process and is a most important aspect in commercial application. This example demonstrates that cannabinoids are suitable in this respect, as they are not miseible in water, spontaneously separating from the biomass and ending-up as floating compounds on the aqueous phase of the reactor and culture that produced them. Such spontaneous product separation from the liquid culture alleviates the requirement of time-consuming, expensive, and technologically complex biomass harvesting and devvafering (Danquah et al., J Chem Tech. Biotech. 84:1078-1083, 2009; Saveyn et al., J Res. Sci Tech. 6:51-56,2009)) and product excision from the cells which otherwise would be needed for product isolation.

In the pursuit of renewable product, photosynthesis, cyanobacteria, or microalgae and cannabinoids meet the above-enumerated criteria for “process”, “organism” and “product”, respectively. This example shows that cannabinoids can be heterologously produced via photosynthesis in microorganisms, e.g., cyanobacteria, genetically engineered to heterologously express plant nptI*GPPS and the cannabinoid biosynthesis operon genes.

The cannabinoids discussed in the present disclosure are useful in, e.g., the cosmetics, biopharmaceutical, and medicinal fields. Currently, cannabinoids are extracted from plants, such as Cannabis which, depending on the species, may contain a variety of cannabinoids and other compounds in their glandular trichome essential oils. However, this example shows that specific and high purity cannabinoids can be produced by photosynthetic microorganisms, e.g., cyanobacteria and microalgae, through heterologous expression of, e.g., the nptI*GPPS and the cannabinoid biosynthesis operon genes in a reaction of the native MEP and heterologous MVA pathway, driven by the process of cellular photosynthesis. Since the carbon atoms used to generate cannabinoids in such a system originate from CO₂, cyanobacterial and microalgal production represents a carbon-neutral source of biopharmaceutical and medicinal compounds. Cannabinoids would also be suitable as a feedstock and building block for the chemical synthesis of alternative biopharmaceutical and medicinal compounds, for use in the respective industries.

### Example 2

### Cyanobacterial Cannabinoid Analysis by GC-MS

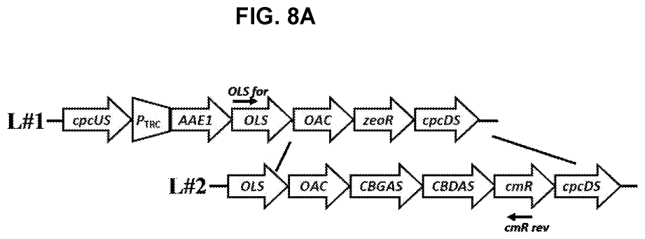

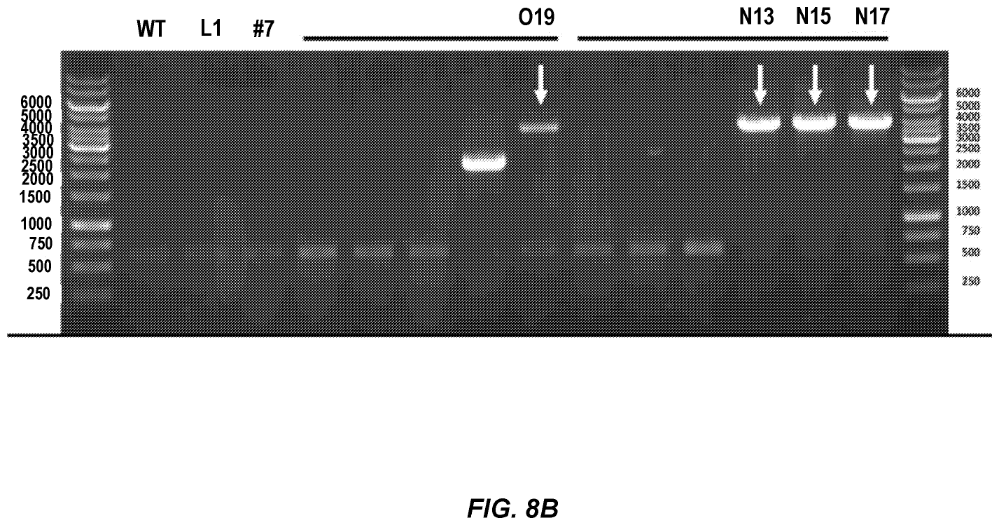

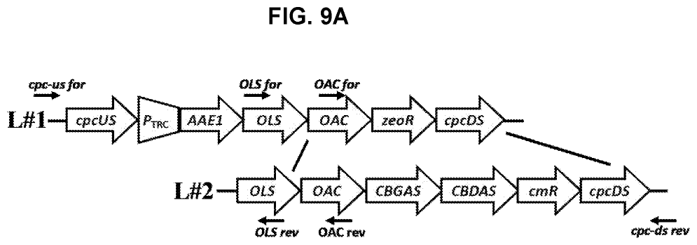

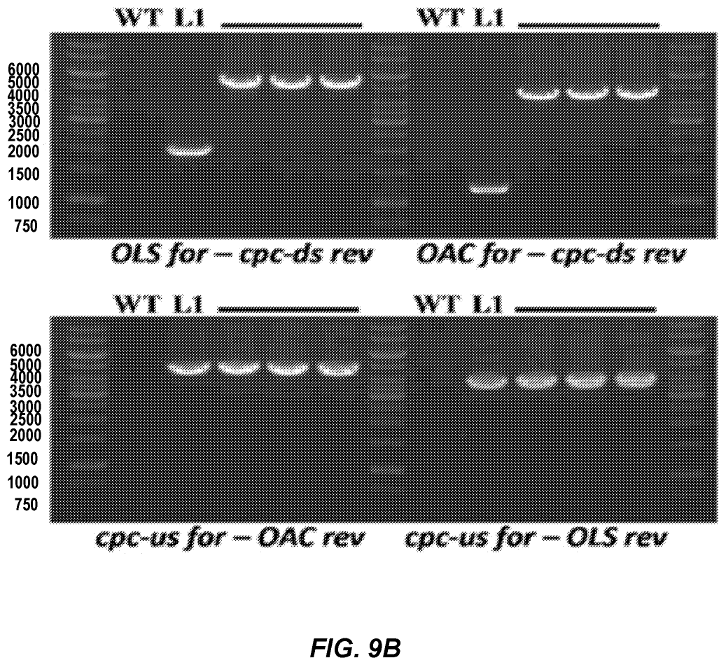

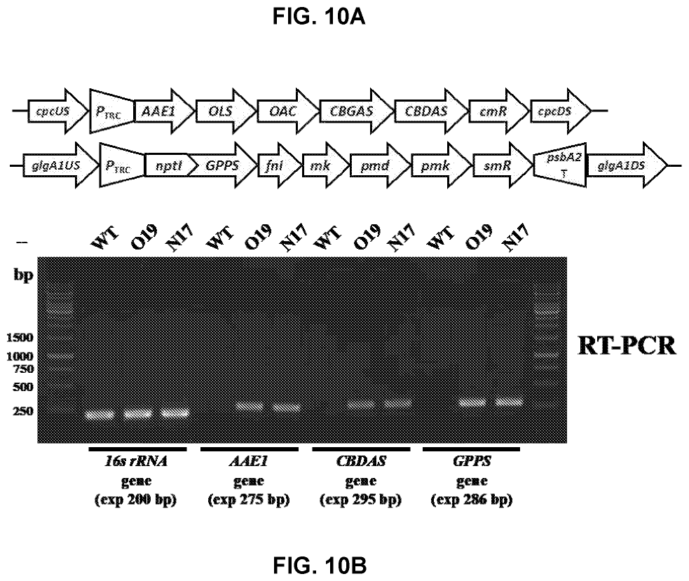

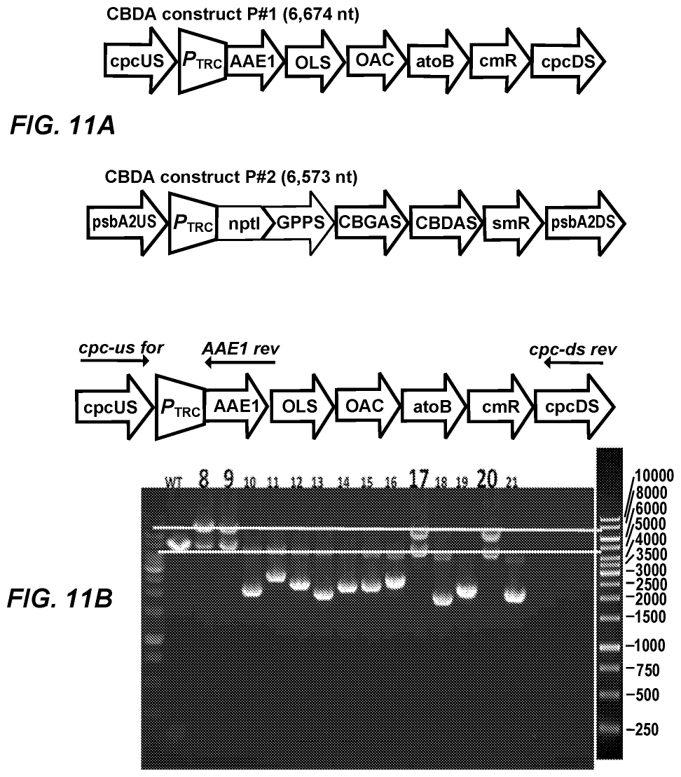

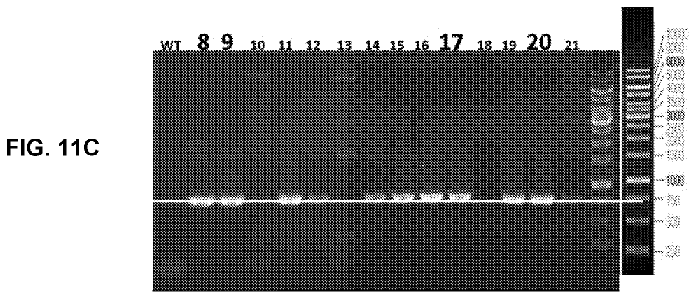

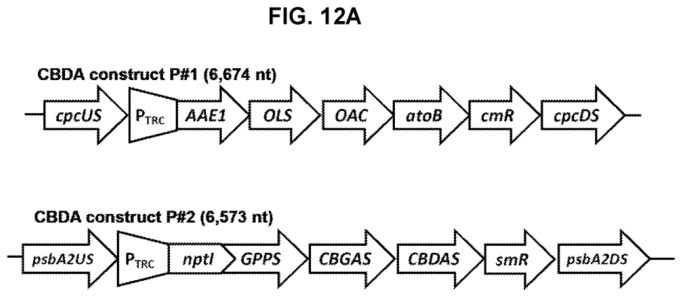

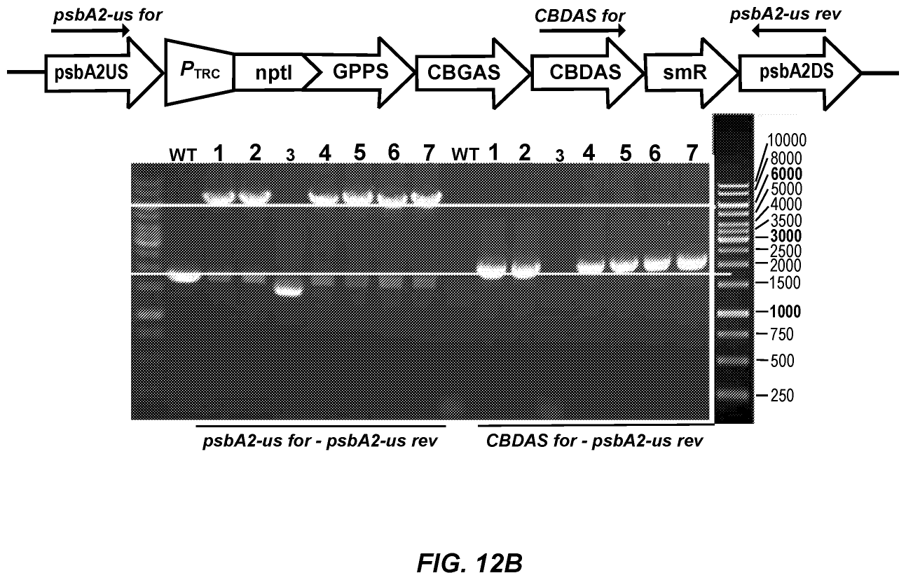

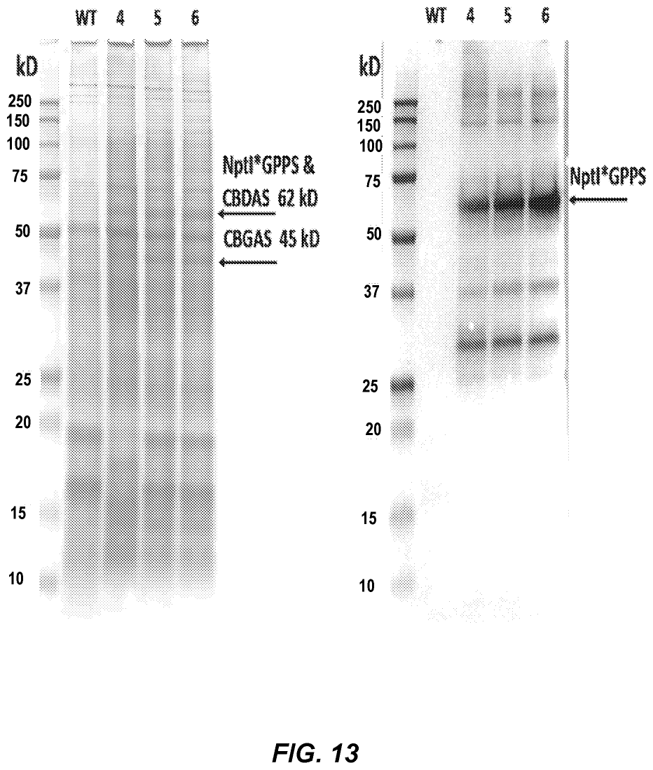

Cyanobacterial cells (Synechocystis) were transformed with genes of the cannabidiolic acid (CBDA) biosynthetic pathway (FIGS. 8-13). Cells were grown in 150 mL liquid media for 3 days. The starting culture OD730 was 0.2. One hundred twenty-five (125) mL were centrifuged at 5000 g for 20 min. The pellet was rcsuspended in 5 mL distilled water. Passage of the cells through French press at 1,500 psi resulted in disintegration of the cells. The crude cell extract was centrifuged at 14,000 g for 5 min to remove large debris and the supernatant was used for cannabinoid extraction, as follows. In a glass vial, 3 mL of the supernatant were mixed with 0.12 mL of H₂SO₄ and 0.5 mL of 30% (w:v) NaCl. This mix was extracted with 3 mL of hexane. The organic layer was separated from the aqueous medium and dried by solvent evaporation. The dry extract was resuspended with 0.1 mL of BSTFA including 1% TMCS (derivatization reagents) and injected in GC-MS for content analysis. CG-MS standards were prepared by drying the original solvent and rcsuspending in BSTFA+1% TMCS prior to injection in the GC-MS. The results, presented in Table 2, showed evidence for the presence of CBDA (most abundant), CBD, Olivetolic acid and Olivetol in the transgenic cell extracts.

TABLE 2

| | | | Cyanobacterial- |
|---|---|---|---|
| | | Main | specific GC-MS |
| | GC-MS retention | GC-MS lines of the | lines identified in |
| Compound | time, min | standard | total cell extracts |
| CBDA | 8.93 | 491, 559, 453 | 491, 559, 453 |
| CBD | 8.05 | 390, 337 | 390, 337 |
| Olivetolic acid | 7.44 | 425 | 425 |
| Olivetol | 6.00 | 268 | 268 |

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of producing a cannabinoid in a photosynthetic microorganism, the method comprising:
(a) introducing into the microorganism:
a polynucleotide encoding a GPPS polypeptide; and
one or more polynucleotides encoding AAE1, OLS, OAC, CBGAS polypeptides, and an oxidocyclase selected from the group consisting of CBDAS, THCAS, and CBCAS; wherein
(i) the polynucleotide encoding the GPPS polypeptide is operably linked to a first promoter; and
(ii) the one or more polynucleotides encoding the AAE1, OLS, OAC, CBGAS polypeptides and the oxidocyclase are operably linked to one or more additional promoters; and
(b) culturing the microorganism under conditions in which GPPS, AAE1, OLS, OAC, CB GAS, and the oxidocyclase are expressed and wherein cannabinoid biosynthesis takes place.

2. The method of claim 1, wherein the photosynthetic microorganism is cyanobacteria.

3. The method of claim 2, wherein the GPPS polypeptide is a fusion protein encoded by a polynucleotide encoding GPPS fused to the 3′ end of a leader nucleic acid sequence encoding a protein that is expressed in cyanobacteria at a level of at least 1% of the total cellular protein.

4. The method of claim 3, wherein the GPPS polypeptide is an nptI*GPPS fusion protein.

5. The method of claim 4, wherein the GPPS polypeptide comprises the amino acid sequence of SEQ ID NO:2, or an amino acid sequence that is at least 90% or 95% identical to SEQ ID NO:2.

6. (canceled)

7. The method of claim 4, wherein the polynucleotide encoding the GPPS polypeptide comprises the nucleotide sequence of SEQ ID NO:1, or a nucleotide sequence that is at least 90% or 95% identical to SEQ ID NO:1.

8. (canceled)

9. The method of claim 1, wherein the AAE1 polypeptide comprises the amino acid sequence of SEQ ID NO:4, or an amino acid sequence that is at least 90% or 95% identical to SEQ ID NO:4.

10. (canceled)

11. The method of claim 9, wherein the polynucleotide encoding the AAE1 polypeptide comprises nucleotides 636-2798 of SEQ ID NO:3, or a nucleotide sequence that is at least 90% or 95% identical to nucleotides 636-2798 of SEQ ID NO:3.

12. (canceled)

13. The method of claim 1, wherein the OLS polypeptide comprises the amino acid sequence of SEQ ID NO:5, or an amino acid sequence that is at least 90% or 95% identical to SEQ ID NO:5.

14. (canceled)

15. The method of claim 13, wherein the polynucleotide encoding the OLS polypeptide comprises nucleotides 2819-3973 of SEQ ID NO:3, or a nucleotide sequence that is at least 90% or 95% identical to nucleotides 2819-3973 of SEQ ID NO:3.

16. (canceled)

17. The method of claim 1, wherein the OAC polypeptide comprises the amino acid sequence of SEQ ID NO:6, or an amino acid sequence that is at least 90% or 95% identical to SEQ ID NO:6.

18. (canceled)

19. The method of claim 17, wherein the polynucleotide encoding the OAC polypeptide comprises nucleotides 3994-4299 of SEQ ID NO:3, or a nucleotide sequence that is at least 90% or 95% identical to nucleotides 3994-4299 of SEQ ID NO:3.

20. (canceled)

21. The method of claim 1, wherein the CBGAS polypeptide comprises the amino acid sequence of SEQ ID NO:7, or an amino acid sequence that is at least 90% or 95% identical to SEQ ID NO:7.

22. (canceled)

23. The method of claim 21, wherein the polynucleotide encoding the CBGAS polypeptide comprises nucleotides 4320-5507 of SEQ ID NO:3, or a nucleotide sequence that is at least 90% or 95% identical to nucleotides 4320-5507 of SEQ ID NO:3.

24. (canceled)

25. The method of claim 1, wherein the oxidocyclase is CBDAS, and wherein the CBDAS comprises the amino acid sequence of SEQ ID NO:8, or an amino acid sequence that is at least 90% or 95% identical to SEQ ID NO:8.

26. (canceled)

27. The method of claim 25, wherein the polynucleotide encoding the CBDAS comprises nucleotides 5528-7162 of SEQ ID NO:3, or a nucleotide sequence that is at least 90% or 95% identical to nucleotides 5528-7162 of SEQ ID NO:3.

28. (canceled)

29. The method of claim 1, wherein the oxidocyclase is THCAS, and wherein the THCAS comprises the amino acid sequence of SEQ ID NO:10, or an amino acid sequence that is at least 90% or 95% identical to SEQ ID NO:10.

30. (canceled)

31. The method of claim 29, wherein the polynucleotide encoding the THCAS comprises the nucleotide sequence of SEQ ID NO:9, or a nucleotide sequence that is at least 90% or 95% identical to SEQ ID NO:9.

32. (canceled)

33. The method of claim 1, wherein the oxidocyclase is CBCAS, and wherein the CBCAS comprises the amino acid sequence of SEQ ID NO:12, or an amino acid sequence that is at least 90% or 95% identical to SEQ ID NO:12.

34. (canceled)

35. The method of claim 33, wherein the polynucleotide encoding the CBCAS comprises the nucleotide sequence of SEQ ID NO:11, or a nucleotide sequence that is at least 90% or 95% identical to SEQ ID NO:11.

36. (canceled)

37. The method of claim 1, wherein two or more of the polynucleotides encoding the AAE1, OLS, OAC, CBGAS polypeptides and the oxidocyclase are present within a single operon.

38-41. (canceled)

42. The method of claim 1, wherein one or more of the polynucleotides encoding the GPPS, AAE1, OLS, OAC, CBGAS polypeptides and the oxidocyclase are codon optimized for the photosynthetic microorganism.

43-44. (canceled)

45. The method of claim 1, further comprising a step
(c) isolating cannabinoids from the microorganism or from the culture medium.

46. The method of claim 45, wherein the cannabinoids are collected from the surface of the liquid culture as floater molecules.

47. The method of claim 45, wherein the cannabinoids are extracted from the interior of the microorganism.

48-56. (canceled)

57. A photosynthetic microorganism produced using the method of claim 1.

58. A photosynthetic microorganism comprising
(a) a polynucleotide encoding a GPPS polypeptide; and
(b) one or more polynucleotides encoding AAE1, OLS, OAC, CBGAS polypeptides and an oxidocyclase selected from the group consisting of CBDAS, THCAS, and CBCAS; wherein
(i) the polynucleotide encoding the GPPS polypeptide is operably linked to a first promoter, and
(ii) the one or more polynucleotides encoding the AAE1, OLS, OAC, CBGAS polypeptides and the oxidocyclase are operably linked to one or more additional promoters.

59. The microorganism of claim 58, wherein the microorganism is cyanobacteria.

60. The microorganism of claim 59, wherein the GPPS polypeptide is a fusion protein encoded by a polynucleotide encoding GPPS fused to the 3′ end of a leader nucleic acid sequence encoding a protein that is expressed in cyanobacteria at a level of at least 1% of the total cellular protein.

61-99. (canceled)

100. The microorganism of claim 58, wherein the microorganism is from a genus selected from the group consisting of Synechocystis, Synechococcus, Athrospira, Nostoc, and Anabaena.

101. (canceled)

102. A polynucleotide encoding GPPS, AAE1, OLS, OAC, CBGAS, CBDAS, THCAS, and/or CBCAS, wherein the polynucleotide is codon optimized for cyanobacteria or another photosynthetic microorganism; and wherein the polynucleotide is at least 90% or 95% identical to a sequence selected from the group consisting of SEQ ID NO:1, SEQ ID NO:3, SEQ ID NO:9, SEQ ID NO:11, SEQ ID NO:13, SEQ ID NO:14, nucleotides 635-2798 of SEQ ID NO:3, nucleotides 2819-3973 of SEQ ID NO:3, nucleotides 3994-4299 of SEQ ID NO:3, nucleotides 4320-5507 of SEQ ID NO:3, and nucleotides 5528-7162 of SEQ ID NO:3.

103. (canceled)

104. An expression cassette comprising the polynucleotide of claim 102.

105. A host cell comprising the expression cassette of claim 104.

106. A cell culture comprising the host cell of claim 105.

107. A method of producing cannabinoids, comprising
culturing the host cell of claim 105, under conditions in which the GPPS, AAE1, OLS, OAC, CBGAS polypeptides and the oxidocyclase are expressed and wherein cannabinoid biosynthesis takes place.

108. The method of claim 107, further comprising isolating cannabinoids from the microorganism or from the culture medium.

109-119. (canceled)
